(19) United States
(12) Patent Application Publication
(10) Pub. No.: US 2024/0350464 A1
(43) Pub. Date: Oct. 24, 2024

(54) METHODS OF TREATING NEUROLOGICAL, MUSCULAR, AND PROLIFERATIVE DISORDERS

(71) Applicant: Elixira Pharma AG, Zug (CH)

(72) Inventors: Alla Zozulya Weidenfeller, Fällenden (CH); Rosa Maria Da Silva Oliveira, Zug (CH); Iryna Fingerhut, Oberägeri (CH)

(21) Appl. No.: 18/637,986

(22) Filed: Apr. 17, 2024

Related U.S. Application Data
(60) Provisional application No. 63/460,487, filed on Apr. 19, 2023.

(51) Int. Cl. A61K 31/436; A61K 31/155; A61P 21/00; A61P 43/00

(52) U.S. Cl. CPC A61K 31/436; A61K 31/155; A61P 21/00; A61P 43/00

(57) ABSTRACT

This disclosure relates to methods of treating neurological, muscular, and proliferative disorders using a combination of metformin and rapamycin.

### CROSS-REFERENCE TO RELATED APPLICATIONS

This application claims priority to U.S. Provisional Application No. 63/460,487, filed on Apr. 19, 2023, which is incorporated by reference herein in its entirety.

### TECHNICAL FIELD

This disclosure relates to methods of treating disorders such as neurological, muscular, and proliferative disorders using a combination of metformin and rapamycin.

### BACKGROUND

The underlying pathophysiology of a number of neurological, muscular, and proliferative disorders (for example, myotonic dystrophy type 1 (DM1), Duchenne muscular dystrophy (DMD), and spinocerebellar ataxia type 3 (SCA-3)) is associated with impaired activation of active adenosine monophosphate-activated protein kinase (AMPK), dysfunction of mammalian target of rapamycin complex 1 (mTORC1), and/or perturbed autophagic flux. There are no known cures for neurological, muscular, and proliferative disorders such as DM1, DMD, and SCA-3 and there are few existing treatments efficacious in treating these disorders with a favorable profile of adverse effects, with many of the treatments limited to ameliorating symptoms to improve the quality of life of patients. Treatments for DM1 include, for example, mexiletine, beta blockers, and angiotensin-converting enzyme (ACE) inhibitors. Treatments for DMD include, for example, prednisone and deflazacort. New treatments for neuromuscular diseases, including gene therapies, small molecule therapies, and RNA-targeted therapies, are still in the experimental stage and require further testing before they can be used in clinical practice.

Thus, there is a need for a method of treating and/or preventing neurological, muscular, and proliferative disorders associated with impaired activation of active adenosine monophosphate-activated protein kinase (AMPK), dysfunction of mammalian target of rapamycin complex 1 (mTORC1), and/or perturbed autophagic flux rather than managing or ameliorating symptoms of such disorders; and where the method has less severe side effects and is conveniently administered as a single dosage form.

### SUMMARY

Described herein are methods of treating or preventing a neurological, muscular, or proliferative disorder in a subject in need thereof, the method including administering a therapeutically effective amount of metformin or a pharmaceutically acceptable salt thereof and rapamycin or a pharmaceutically acceptable salt thereof to the subject; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

In some embodiments, the neurological, muscular, or proliferative disorder is myotonic dystrophy type 1 (DM1). In some embodiments, after administering the metformin or a pharmaceutically acceptable salt thereof and rapamycin or a pharmaceutically acceptable salt thereof, a reduction in reactive oxygen species (ROS) is measured in the subject.

In some embodiments, the neurological, muscular, or proliferative disorder is Duchenne muscular dystrophy (DMD).

In some embodiments, the neurological, muscular, or proliferative disorder is spinocerebellar ataxia type 3 (SCA-3).

In some embodiments, the neurological, muscular, or proliferative disorder is Alzheimer's disease.

In some embodiments, the neurological, muscular, or proliferative disorder is Parkinson's disease.

In some embodiments, the neurological, muscular, or proliferative disorder is vascular dementia.

In some embodiments, the neurological, muscular, or proliferative disorder is dementia with Lewy bodies (DLB).

In some embodiments, the neurological, muscular, or proliferative disorder is Huntington disease (HD).

In some embodiments, the neurological, muscular, or proliferative disorder is amyotrophic lateral sclerosis (ALS).

In some embodiments, the neurological, muscular, or proliferative disorder is Lafora disease.

In some embodiments, the method includes preventing the disorder.

In some embodiments, the neurological, muscular, or proliferative disorder is neuroglioblastoma.

In some embodiments, the neurological, muscular, or proliferative disorder is diffuse intrinsic pontine glioma (DIPG). In some embodiments, after administering the metformin or a pharmaceutically acceptable salt thereof and rapamycin or a pharmaceutically acceptable salt thereof, Akt is activated in the subject.

Some embodiments provide a method of treating or preventing a neurological cancer in a subject in need thereof, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG)).

In some embodiments, the neurological cancer is neuroglioblastoma.

In some embodiments, the neurological cancer is diffuse intrinsic pontine glioma (DIPG). In some embodiments, after administering the metformin or a pharmaceutically acceptable salt thereof and rapamycin or a pharmaceutically acceptable salt thereof, Akt is activated in the subject.

Some embodiments provide a method of increasing muscular strength in a subject identified or diagnosed as having muscular weakness, the method including administering a therapeutically effective amount of metformin or a pharmaceutically acceptable salt thereof and rapamycin or a pharmaceutically acceptable salt thereof to the subject.

Some embodiments provide a method of preventing or reversing muscle weakness in a subject in need thereof, the method including administering a therapeutically effective amount of metformin or a pharmaceutically acceptable salt thereof and rapamycin or a pharmaceutically acceptable salt thereof to the subject. In some embodiments, after administering the therapeutically effective amount of metformin or a pharmaceutically acceptable salt thereof and rapamycin or a pharmaceutically acceptable salt thereof to the subject, an increase muscle mass using magnetic resonance imaging (MRI) assessment is measured in the subject.

In some embodiments, the method includes preventing cellular senescence in the subject.

Some embodiments provide a method of increasing motility in a subject, comprising administering a therapeutically effective amount of metformin or a pharmaceutically acceptable salt thereof and rapamycin or a pharmaceutically acceptable salt thereof to the subject.

In some embodiments, the method includes increasing the number of voluntary muscle contractions in the subject per minute relative to before the therapeutically effective amount of metformin or a pharmaceutically acceptable salt thereof and rapamycin or a pharmaceutically acceptable salt thereof was administered to the subject.

In some embodiments, the method includes increasing the duration of a voluntary muscle contraction in the subject relative to before the therapeutically effective amount of metformin or a pharmaceutically acceptable salt thereof and rapamycin or a pharmaceutically acceptable salt thereof was administered to the subject.

In some embodiments, the method includes determining an increase in the distance walked by the subject during a 10 meter walk test after administering the metformin or a pharmaceutically acceptable salt thereof and rapamycin or a pharmaceutically acceptable salt thereof.

In some embodiments, the method includes determining a reduction in the time taken by the subject to complete a 100 meter walk test after administering the metformin or a pharmaceutically acceptable salt thereof and rapamycin or a pharmaceutically acceptable salt thereof.

In some embodiments, after administering the metformin or a pharmaceutically acceptable salt thereof and rapamycin or a pharmaceutically acceptable salt thereof, an increase in AMPK activation, inhibition of mTORC1, inhibition of S6 kinase, or any combination thereof are determined in the subject.

In some embodiments, the metformin or a pharmaceutically acceptable salt thereof and rapamycin or a pharmaceutically acceptable salt thereof are administered orally. In some embodiments, the metformin or a pharmaceutically acceptable salt thereof is administered daily.

In some embodiments, the dose of metformin or a pharmaceutically acceptable salt thereof is from about 500 mg to about 3000 mg on a free base basis of metformin. In some embodiments, the dose of metformin or a pharmaceutically acceptable salt thereof is from about 500 to about 1750 mg on a free base basis of metformin.

In some embodiments, the rapamycin or a pharmaceutically acceptable salt thereof is administered daily.

In some embodiments, the dose of rapamycin or a pharmaceutically acceptable salt thereof is from about 0.1 mg to about 2 mg on a free base basis of rapamycin. In some embodiments, the dose of rapamycin or a pharmaceutically acceptable salt thereof is from about 0.5 mg to about 1 mg on a free base basis of rapamycin. In some embodiments, the dose of rapamycin or a pharmaceutically acceptable salt thereof is about 0.7 mg on a free base basis of rapamycin.

In some embodiments, the metformin or a pharmaceutically acceptable salt thereof and rapamycin or a pharmaceutically acceptable salt thereof are administered simultaneously. In some embodiments, the metformin or a pharmaceutically acceptable salt thereof and rapamycin or a pharmaceutically acceptable salt thereof are administered simultaneously as a fixed dosage form.

In some embodiments, the rapamycin or a pharmaceutically acceptable salt thereof is administered weekly.

In some embodiments, the dose of rapamycin or a pharmaceutically acceptable salt thereof is from about 1 mg to about 10 mg on a free base basis of rapamycin. In some embodiments, the dose of rapamycin or a pharmaceutically acceptable salt thereof is from about 2 mg to about 10 mg on a free base basis of rapamycin. In some embodiments, the dose of rapamycin or a pharmaceutically acceptable salt thereof is about 5 mg on a free base basis of rapamycin.

In some embodiments, a cyclosporine, tacrolimus, and mycophenolate mofetil were not previously administered to the subject within 1 month of administering the metformin or a pharmaceutically acceptable salt thereof and rapamycin or a pharmaceutically acceptable salt thereof. In some embodiments, the subject is not identified or diagnosed as having a disease associated with a kidney. In some embodiments, the subject is not identified or diagnosed as having a disease associated with the liver. In some embodiments, the subject is not identified or diagnosed as having a disease associated with the heart. In some embodiments, the subject is not identified or diagnosed as having diabetes. In some embodiments, the subject is not identified or diagnosed as having abnormal endocrine function. In some embodiments, the subject was not previously administered a therapeutic agent that modulates the insulin transduction pathway within 1 year of administering the metformin or a pharmaceutically acceptable salt thereof and rapamycin or a pharmaceutically acceptable salt thereof.

The details of one or more embodiments of the invention are set forth in the description below. Other features and advantages of the invention will be apparent from the description and drawings, and from the claims.

### BRIEF DESCRIPTION OF DRAWINGS

FIG. 1 is an image depicting the design and timeline of a study of the effect of rapamycin, metformin, and their combination on lifespan and healthspan on C. elegans.

FIG. 2 is a series of survival curves of N2 wild type worms treated with rapamycin, metformin, combination A, combination B, combination C, and combination D.

FIG. 3 is a series of survival curves to day 17.5 (corresponding to ˜50% survival of the negative control DMSO 1%) of N2 wild type worms treated with rapamycin, metformin, combination A, combination B, combination C, and combination D.

FIG. 4 is a series of survival curves to day 20 (corresponding to ˜25% survival of the negative control DMSO 1%) of N2 wild type worms treated with rapamycin, metformin, combination A, combination B, combination C, and combination D.

FIGS. 5A-5D are bar graphs of the time (in hours) from injection of the L4 larvae until production of the first (FIG. 5A) and last (FIG. 5B) egg, as well as the timespan of egg laying (FIG. 5C) and the average number of eggs laid per worm within the timespan of egg laying (FIG. 5D), observed in N2 wild type worms treated with rapamycin, metformin, combination A, combination B, combination C, and combination D.

FIG. 6 is a bar graph of the worm's growth (area under the curves) of N2 wild type worms treated with rapamycin, metformin, combination A, combination B, combination C, and combination D.

FIGS. 7A-7D are plots of worm's motility (FIG. 7A, amplitude of the head; FIG. 7B, amplitude of the midbody; FIG. 7C, amplitude of the tail; FIG. 7D, bending frequency; FIG. 7E, velocity) of N2 wild type worms treated with rapamycin, metformin, combination A, combination B, combination C, and combination D.

FIGS. 8A-8C are radar charts of worm's motility (amplitude of the head, amplitude of the midbody, amplitude of the tail, velocity and bending frequency) at different phases (1: D0 to D5; 2: D6 to D10; 3: D11 to D15; 4: D16-D20) of N2 wild type worms treated with rapamycin and metformin (FIG. 8A); combination A and combination B (FIG. 8B); and combination C and combination D (FIG. 8C).

### DETAILED DESCRIPTION

Described herein are methods of (i) treating and/or preventing neurological, muscular, or proliferative disorders, diseases, or conditions, and (ii) methods of reducing and/or preventing processes associated with aging (such as cellular senescence) including administering metformin or a pharmaceutically acceptable salt thereof in combination with rapamycin or a pharmaceutically acceptable salt thereof. AMP-activated protein kinase (AMPK) and the mammalian target of rapamycin (mTOR) pathway are two signaling pathways that play roles in cellular metabolism, growth, and proliferation. AMPK activation and mTOR inhibition can, for example, (1) protect neurons from damage and improve cognitive function, thus being a potential target for the treatment of neurodegenerative disorders such as Alzheimer's disease and Parkinson's disease; (2) enhance muscle protein synthesis, thus being a potential therapy for muscular disorders such as muscular dystrophy and myotonic dystrophy; and (3) suppress tumor growth and induce cancer cell death, thus being a potential strategy for cancer therapy. Surprisingly, the inventors have discovered that metformin (which, e.g., activates AMPK) and rapamycin (which e.g., inhibits mTORC1) synergistically treat the aforementioned disorders, thus reducing the dose required and therefore adverse effects from targeting of undesired metabolic pathways. Additional benefits include, for example, decreasing concentrations of insulin and insulin growth factor 1 (IGF-1), inducing energetic stress, restored cerebral blood flow (CBF) and brain vascular density, reduced cerebral amyloid angiopathy and microhemorrhages, reducing inflammatory cytokines (e.g., IL-6), reducing pro-inflammatory T helper cell 1 (Th1) and T helper cell 17 (Th17). Further, the safety and efficacy profiles of metformin and rapamycin have been extensively studied. Without wishing to be bound by theory, the two agents are believed to have a complementary profile of side effects. For example, it is believed that metformin can reduce the risk of hyperglycemia and hyperlipidemia caused by rapamycin, while rapamycin can reduce the gastrointestinal side effects of metformin. In addition, the combination of metformin and rapamycin can have synergistic effects on health and lifespan.

### Definitions

As used herein, the terms “about” and “approximately” are used interchangeably, and when used to modify a numerical value, encompass a range of uncertainty of the numerical value of from 0% to 10% of the numerical value.

As used herein, the singular forms “a,” “an,” and “the” include plural referents unless the context clearly dictates otherwise.

As used herein, the terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “preventing” as used herein means the prevention of the onset, recurrence or spread, in whole or in part, of the disease or condition as described herein, or a symptom thereof. The term “preventive measure” refers to an action performed that prevents the onset, recurrence or spread, in whole or in part, of the disease or condition as described herein.

As used herein, the terms “subject,” “individual,” and “patient” are used interchangeably, and refer to any animal, including mammals such as mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

As used herein, the term “adverse effect” refers to an undesirable effect resulting from an alteration in normal physiology in a subject.

The term “pharmaceutical composition” as used herein is intended to encompass a product including the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt thereof, and a pharmaceutically acceptable carrier.

The term “pharmaceutically acceptable carrier” or “pharmaceutically acceptable excipient” refers to a carrier or an adjuvant that may be administered to a patient, together with a compound of the present disclosure, or a pharmaceutically acceptable salt thereof, and which does not destroy the pharmacological activity thereof and is nontoxic when administered in doses sufficient to deliver a therapeutic amount of the compound. In some embodiments, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

As used herein, the phrase “fixed dosage form” refers to the simultaneous administration of two or more therapeutic agents together in a single dosage form (e.g., in a single oral dosage form such as a pill, tablet, or capsule). In this context, when metformin or a pharmaceutically acceptable salt thereof and rapamycin or a pharmaceutically acceptable salt thereof are administered as a fixed dosage form, both drugs are administered together in a single dosage form (e.g., an oral dosage form such as, e.g., a pill, tablet, or capsule) containing both metformin or a pharmaceutically acceptable salt thereof and rapamycin or a pharmaceutically acceptable salt thereof. In some embodiments, a fixed dosage form can include metformin, rapamycin, and one or more additional therapeutic agents.

The term “abnormally” when used, for example, in the terms “abnormally high,” “abnormally elevated,” or “abnormally low,” means a deviation from the range of the parameter being referred to that is found in a healthy subject as would be recognized by a medical professional (e.g., a doctor, nurse practitioner, medical laboratory scientist, nurse practitioner, physical therapist, or physician assistant), and that can be considered as indicative or predictive of dysfunction or a pathological state. Further, “abnormal” can, in some embodiments, refer to a physiologic response that is persistent beyond when a normal person would have recovered from that response; or a physiologic response that is exaggerated in degree and/or duration relative to what occurs in a normal, healthy subject.

As used herein, “autophagy” refers to the natural, conserved degradation of a cell that degrades, removes, and recycles unnecessary or dysfunctional components of the cell. In some embodiments, promoting autophagy in the subject includes increasing the frequency of the number of cells and/or cell components undergoing autophagy and/or autophagic processes in the subject.

The term “therapeutically effective amount,” as used herein, refers to the amount of one or more active chemical entities or pharmaceutical agent (e.g., metformin and rapamycin) being administered which elicits the biological or medicinal response in a tissue, system, animal, individual, or human that is being sought. In some embodiments, the response includes reduction and/or alleviation of the signs, symptoms, or causes of a disease, or any other desired alteration of a biological system. An appropriate “therapeutically effective amount” in any individual case is determined, e.g., using any suitable technique, such as a dose escalation study.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The term “pharmaceutically acceptable salt” refers to a salt formed by the addition of a pharmaceutically acceptable acid or base to a compound disclosed herein. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” can also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salts are not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts can be acid addition salts, which are specifically exemplified by acid addition salts with the following: mineral acids such as hydrochloric acid, hydrobromic acid, hydroiodic acid, sulfuric acid, nitric acid, and phosphoric acid; organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; and acidic amino acids such as aspartic acid and glutamic acid.

For purposes of clarification, unless otherwise specified herein, when a variable (e.g., condition, feature, state, parameter, score, assessment, test, or statistic) in a subject is increased, decreased, or improved, the increase, decrease, or improvement is, for example, measured, assessed, or obtained in relation to the same variable measured, assessed, or obtained before the start of treatment (e.g., before administration of the metformin or a pharmaceutically acceptable salt thereof and the rapamycin or a pharmaceutically acceptable salt thereof), unless otherwise specified herein. The variable can be a single measurement, assessment, or score; an average of a plurality of measurements, assessments, or scores; or a daily average of a plurality of measurements, scores, or assessments. Unless otherwise specified herein, measurements, assessments, or scores are typically taken within 1 month (e.g., within 3 weeks, 2 weeks, 10 days, 9 days, 8 days, 7 days, 6 days, 5 days, 4 days, 3 days, 2 days, 1 day, 18 hours, 12 hours, 6 hours, 3 hours, 2 hours, 1 hour, 30 minutes, or 15 minutes) of the administration of the metformin and/or rapamycin (e.g., the metformin and rapamycin). For example, reducing the frequency of symptoms in a subject can occur, e.g., when the number or average number of symptomatic episodes perceived by the subject that occurred during a span of time after administration of the metformin and/or rapamycin (e.g., the metformin and rapamycin) is less than the number or average number of symptomatic episodes perceived by the subject that occurred during the same span of time before administration of the metformin and/or rapamycin (e.g., the metformin and rapamycin).

Provided in the present disclosure is a method of treating or preventing a neurological, muscular, or proliferative disorder, disease, or condition in a subject in need thereof. In some embodiments, the method includes administering a therapeutically effective amount of metformin or a pharmaceutically acceptable salt thereof and rapamycin or a pharmaceutically acceptable salt thereof to the subject. In some embodiments, the neurological, muscular, or proliferative disorder is selected from the group containing: myotonic dystrophy type 1 (DM1), Duchenne muscular dystrophy (DMD), spinocerebellar ataxia type 3 (SCA-3, i.e., Machado-Joseph disease), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

In some embodiments, the method includes determining if the disorder is associated with an impaired activation of active adenosine monophosphate-activated protein kinase (AMPK). In some embodiments, the method includes determining if the disorder is associated with dysfunction of mammalian target of rapamycin complex 1 (mTORC1). In some embodiments, the method includes determining if the disorder is associated with perturbed autophagic flux. In some embodiments, if the disorder is determined to be associated with impaired activation of active adenosine monophosphate-activated protein kinase (AMPK), overactivation of mammalian target of rapamycin complex 1 (mTORC1), and/or perturbed autophagic flux, the method includes administering metformin or a pharmaceutically acceptable salt thereof and rapamycin or a pharmaceutically acceptable salt thereof to the subject.

Some embodiments provide a method of treating or preventing a neurological, muscular, or proliferative disorder, disease, or condition in a subject in need thereof, the method including administering a therapeutically effective amount of metformin or a pharmaceutically acceptable salt thereof and rapamycin or a pharmaceutically acceptable salt thereof to the subject, where the neurological, muscular, or proliferative disorder is selected from the group containing: myotonic dystrophy type 1 (DM1), Duchenne muscular dystrophy (DMD), spinocerebellar ataxia type 3 (SCA-3, i.e., Machado-Joseph disease), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of treating a neurological, muscular, or proliferative disorder in a subject in need thereof, the method including administering a therapeutically effective amount of metformin or a pharmaceutically acceptable salt thereof and rapamycin or a pharmaceutically acceptable salt thereof to the subject; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of preventing a neurological, muscular, or proliferative disorder in a subject in need thereof, the method including administering a therapeutically effective amount of metformin or a pharmaceutically acceptable salt thereof and rapamycin or a pharmaceutically acceptable salt thereof to the subject; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method for treating or preventing a neurological, muscular, or proliferative disorder in a subject in need thereof, the method including (a) determining if the disorder is associated with an impaired activation of active adenosine monophosphate-activated protein kinase (AMPK), dysfunction of mammalian target of rapamycin complex 1 (mTORC1), and/or perturbed autophagic flux; and (b) if the disorder is determined to be associated with impaired activation of active adenosine monophosphate-activated protein kinase (AMPK), overactivation of mammalian target of rapamycin complex 1 (mTORC1), and/or perturbed autophagic flux, administering metformin or a pharmaceutically acceptable salt thereof and rapamycin or a pharmaceutically acceptable salt thereof to the subject; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

In some embodiments, dysfunction of mammalian target of rapamycin complex 1 (mTORC1) includes overactivation of mammalian target of rapamycin complex 1 (mTORC1).

In some embodiments, the subject has a neurological, muscular, or proliferative disorder selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of treating or preventing a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux in a subject, the method including:
determining if a neurological, muscular, or proliferative disorder is associated with an impaired activation of active adenosine monophosphate-activated protein kinase (AMPK), dysfunction of mammalian target of rapamycin complex 1 (mTORC1), and/or perturbed autophagic flux; and
administering to a subject determined to have a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux a therapeutically effective amount of metformin or a pharmaceutically acceptable salt thereof and rapamycin or a pharmaceutically acceptable salt thereof; where the neurological, muscular, proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of treating or preventing a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux in a subject, the method including administering to a subject identified or diagnosed as having an neurological or neuromuscular disorder associated with an impaired activation of active adenosine monophosphate-activated protein kinase (AMPK), dysfunction of mammalian target of rapamycin complex 1 (mTORC1), and/or perturbed autophagic flux a therapeutically effective amount of metformin or a pharmaceutically acceptable salt thereof and rapamycin or a pharmaceutically acceptable salt thereof; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of selecting a treatment or preventive measure for a subject having a neurological, muscular, or proliferative disorder, the method including:
determining if the disorder in the subject is a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux; and selecting a treatment or preventive measure including administration of a therapeutically effective amount of metformin or a pharmaceutically acceptable salt thereof and rapamycin or a pharmaceutically acceptable salt thereof, for the subject; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of selecting a subject for treatment or prevention including administration of a therapeutically effective amount of metformin or a pharmaceutically acceptable salt thereof and rapamycin or a pharmaceutically acceptable salt thereof, the method including:
identifying a subject having a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux; and
selecting the subject for treatment or prevention including administration of a therapeutically effective amount of metformin or a pharmaceutically acceptable salt thereof and rapamycin or a pharmaceutically acceptable salt thereof; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of selecting a treatment or preventive measure for a subject, the method including selecting a treatment or preventive measure including administration of a therapeutically effective amount of metformin or a pharmaceutically acceptable salt thereof and rapamycin or a pharmaceutically acceptable salt thereof, for a subject identified or diagnosed as having a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Some embodiments provide a method of selecting a subject having a neurological, muscular, or proliferative disorder for treatment including administration of metformin or a pharmaceutically acceptable salt thereof and rapamycin or a pharmaceutically acceptable salt thereof, the method including:
determining if the disorder in the subject is a neurological, muscular, or proliferative disorder associated with impaired activation of AMPK and/or overactivation of mTORC1; and
selecting a subject determined to have a neurological, muscular, or proliferative disorder associated with an impaired activation of active adenosine monophosphate-activated protein kinase (AMPK), dysfunction of mammalian target of rapamycin complex 1 (mTORC1), and/or perturbed autophagic flux for treatment including administration of metformin or a pharmaceutically acceptable salt thereof and rapamycin or a pharmaceutically acceptable salt thereof; where the neurological, muscular, or proliferative disorder is selected from the group containing: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

In some embodiments, the method includes selecting a treatment for the subject. In some embodiments, the method includes selecting a preventive measure for the subject.

In some embodiments, the neurological, muscular, or proliferative disorder is selected from the group containing: myotonic dystrophy type 1 (DM1), Duchenne muscular dystrophy (DMD), and spinocerebellar ataxia type 3 (SCA-3).

In some embodiments, the neurological, muscular, or proliferative disorder is selected from the group containing: Alzheimer's disease, Parkinson's disease, vascular dementia, dementia with Lewy bodies (DLB), Huntington disease (HD), amyotrophic lateral sclerosis (ALS), and Lafora disease.

In some embodiments, the neurological, muscular, or proliferative disorder is selected from the group containing: neuroglioblastoma and diffuse intrinsic pontine glioma (DIPG).

In some embodiments, the method includes treating the disorder. In some embodiments, the method includes preventing the disorder.

In some embodiments, the method includes treating one or more symptoms. In some embodiments, the method includes preventing one or more symptoms.

In some embodiments, the neurological, muscular, or proliferative disorder is myotonic dystrophy type 1 (DM1). In some embodiments, treating or preventing the myotonic dystrophy type 1 (DM1) includes treating or preventing one or more symptoms of myotonic dystrophy type 1 (DM1). In some embodiments, the one or more symptoms is selected from the group containing: muscle weakness, muscle atrophy, myotonia, muscle pain, ptosis, low blood oxygen saturation, intellectual impairment, a behavioral disorder, fatigue, a cataract, retinal damage, difficulty breathing, dyspnea, diabetes (e.g., Type I or Type II diabetes), sleep apnea, pneumonia, low testosterone, pilomatrixomas, hypogammalobulinemia, erectile dysfunction, testicular failure, gonadal atrophy, arrhythmia, cardiomyopathy, difficulty swallowing, abdominal pain, irritable bowel syndrome (IBS), constipation, and diarrhea.

In some embodiments, the symptom is muscle weakness.

In some embodiments, the symptom is muscle atrophy.

In some embodiments, the symptom is myotonia.

In some embodiments, the symptom is muscle pain.

In some embodiments, the symptom is ptosis.

In some embodiments, the symptom is low blood oxygen saturation. In some embodiments, low blood oxygen saturation is a blood oxygen saturation of less than 95% (e.g., less than 94%, less than 93%, less than 92%, less than 91%, less than 90%, less than 89%, less than 88%, less than 87%, less than 86%, or less than 85%).

In some embodiments, the symptom is intellectual impairment. In some embodiments, intellectual impairment includes an abnormally low attention span. In some embodiments, intellectual impairment includes impairment in visuospatial skills.

In some embodiments, the symptom is a behavioral disorder. In some embodiments, the behavioral disorder is autism spectrum disorder.

In some embodiments, the symptom is fatigue. In some embodiments, the fatigue includes excessive daytime sleepiness.

In some embodiments, the symptom is a cataract.

In some embodiments, the symptom is retinal damage.

In some embodiments, the symptom is difficulty breathing.

In some embodiments, the symptom is dyspnea.

In some embodiments, the symptom is diabetes (e.g., Type I or Type II diabetes).

In some embodiments, the symptom is sleep apnea.

In some embodiments, the symptom is pneumonia.

In some embodiments, the symptom is low testosterone. In some embodiments, low testosterone is a total testosterone in ng/dL corresponding to lower than 50^{th} percentile (e.g., lower than 45^{th} percentile, lower than 40^{th} percentile, lower than 35^{th} percentile, lower than 30 percentile, lower than 25^{th} percentile, lower than 20^{th} percentile, lower than 15^{th} percentile, lower than 10^{th} percentile, or lower than 5^{th} percentile) among men having the same age or in the same age group.

In some embodiments, the symptom is pilomatrixomas.

In some embodiments, the symptom is hypogammalobulinemia.

In some embodiments, the symptom is erectile dysfunction.

In some embodiments, the symptom is testicular failure.

In some embodiments, the symptom is gonadal atrophy.

In some embodiments, the symptom is arrhythmia.

In some embodiments, the symptom is cardiomyopathy.

In some embodiments, the symptom is difficulty swallowing.

In some embodiments, the symptom is abdominal pain.

In some embodiments, the symptom is irritable bowel syndrome (IBS).

In some embodiments, the symptom is constipation.

In some embodiments, the symptom is diarrhea.

In some embodiments, after administering the metformin or a pharmaceutically acceptable salt thereof and rapamycin or a pharmaceutically acceptable salt thereof, a reduction in reactive oxygen species (ROS) is measured in the subject.

In some embodiments, the reduction in reactive oxygen species (ROS) is measured in a tissue sample of a subject. In some embodiments, the reduction in reactive oxygen species (ROS) is measured in the blood of a subject.

Various methods are known in the art for measuring reactive oxygen species in a subject, including, but not limited to, electron spin resonance (ESR) and spectrophotometry.

In some embodiments, the reduction in reactive oxygen species (ROS) is measured by electron spin resonance (ESR). In some embodiments, the reduction in reactive oxygen species (ROS) is measured by spectrophotometry.

In some embodiments, the neurological, muscular, or proliferative disorder is Duchenne muscular dystrophy (DMD).

In some embodiments, treating or preventing the Duchenne muscular dystrophy (DMD) includes treating or preventing one or more symptoms of Duchenne muscular dystrophy (DMD). In some embodiments, the one or more symptoms is selected from the group containing: muscle weakness, muscle atrophy, difficulty walking, impaired ability to perform routine tasks, falling, a learning disability, enlarged calves, fatigue, poor motor skills, difficulty breathing, and lumbar lordosis.

In some embodiments, the symptom is muscle weakness.

In some embodiments, the symptom is muscle atrophy.

In some embodiments, the symptom is difficulty walking. In some embodiments, difficulty walking includes ataxia, vertigo, poor balance, or dizziness.

In some embodiments, the symptom is impaired ability to perform routine tasks.

In some embodiments, the symptom is falling. In this context, falling is considered an abnormal frequency of falling that does not occur in otherwise healthy individuals (as determined by, for example, a medical professional (e.g., a doctor, nurse practitioner, nurse, or medical technician)).

In some embodiments, the symptom is a learning disability. In some embodiments, the learning disability is dyslexia. In some embodiments, the learning disability is dyscalculia. In some embodiments, the learning disability is dysgraphia.

In some embodiments, the symptom is enlarged calves.

In some embodiments, the symptom is fatigue. In some embodiments, the fatigue includes excessive daytime sleepiness.

In some embodiments, the symptom is poor motor skills.

In some embodiments, the symptom is difficulty breathing.

In some embodiments, the symptom is lumbar lordosis.

In some embodiments, the neurological, muscular, or proliferative disorder is spinocerebellar ataxia type 3 (SCA-3).

In some embodiments, treating or preventing the spinocerebellar ataxia type 3 (SCA-3) includes treating or preventing one or more symptoms of spinocerebellar ataxia type 3 (SCA-3). In some embodiments, the one or more symptoms is selected from the group containing: involuntary eye movements, poor hand-eye coordination, poor balance and coordination, slurred speech, learning disabilities, impaired memory, and uncoordinated walking.

In some embodiments, the symptom is involuntary eye movements.

In some embodiments, the symptom is poor hand-eye coordination.

In some embodiments, the symptom is poor balance and coordination.

In some embodiments, the symptom is slurred speech.

In some embodiments, the symptom is a learning disability.

In some embodiments, the symptom is impaired memory.

In some embodiments, the symptom is uncoordinated walking.

In some embodiments, the neurological, muscular, or proliferative disorder is Alzheimer's disease.

In some embodiments, treating or preventing the Alzheimer's disease includes treating or preventing one or more symptoms of Alzheimer's disease. In some embodiments, the one or more symptoms is selected from the group containing: memory loss, inflexibility and hesitance to try new things, confusion, disorientation, obsessive behavior, repetitive behavior, impulsive behavior, delusions, speech impairment, aphasia, disturbed sleep, frequent and abnormal mood swings, depression, anxiety, frustration, difficulty in performing spatial tasks, difficulty judging distances, agnosia, difficulty in changing position or moving around without assistance, weight loss, weight gain, and loss of ability to speak.

In some embodiments, the symptom is memory loss. In some embodiments, the symptom is short-term memory loss. In some embodiments, the symptom is long-term memory loss. In some embodiments, the memory loss includes misplacing items, forgetting the names of places and objects, repeating things regularly, and asking the same question several times.

In some embodiments, the symptom is inflexibility and hesitance to try new things.

In some embodiments, the symptom is confusion.

In some embodiments, the symptom is disorientation.

In some embodiments, the symptom is obsessive behavior.

In some embodiments, the symptom is repetitive behavior.

In some embodiments, the symptom is impulsive behavior.

In some embodiments, the symptom is delusions.

In some embodiments, the symptom is speech impairment.

In some embodiments, the symptom is aphasia.

In some embodiments, the symptom is disturbed sleep.

In some embodiments, the symptom is frequent and abnormal mood swings.

In some embodiments, the symptom is depression.

In some embodiments, the symptom is anxiety.

In some embodiments, the symptom is frustration.

In some embodiments, the symptom is difficulty in performing spatial tasks.

In some embodiments, the symptom is difficulty judging distances.

In some embodiments, the symptom is agnosia.

In some embodiments, the symptom is difficulty in changing position or moving around. In some embodiments, the symptom is without assistance.

In some embodiments, the symptom is weight loss.

In some embodiments, the symptom is weight gain.

In some embodiments, the symptom is loss of ability to speak.

In some embodiments, the method includes determining that the subject has an abnormal amount of extracellular amyloid plaques and/or tau proteins in the brain before administering the metformin or a pharmaceutically acceptable salt thereof and rapamycin or a pharmaceutically acceptable salt thereof. In some embodiments, the method includes determining that the subject has an abnormal amount of extracellular amyloid plaques in the brain before administering the metformin or a pharmaceutically acceptable salt thereof and rapamycin or a pharmaceutically acceptable salt thereof. In some embodiments, the method includes determining that the subject has an abnormal amount of tau proteins in the brain before administering the metformin or a pharmaceutically acceptable salt thereof and rapamycin or a pharmaceutically acceptable salt thereof. In some embodiments, the method includes determining the extracellular amyloid plaques and/or tau proteins in the brain are reduced after administering the metformin or a pharmaceutically acceptable salt thereof and rapamycin or a pharmaceutically acceptable salt thereof.

In some embodiments, the method includes determining that the subject has an abnormal amount of intracellular neurofibrillary tangles in the brain before administering the metformin or a pharmaceutically acceptable salt thereof and rapamycin or a pharmaceutically acceptable salt thereof. In some embodiments, the method includes determining that the intracellular neurofibrillary tangles in the brain are reduced after administering the metformin or a pharmaceutically acceptable salt thereof and rapamycin or a pharmaceutically acceptable salt thereof.

In some embodiments, determining the amount of extracellular amyloid plaques, tau proteins, and/or intracellular neurofibrillary tangles in the brain includes performing an imaging technique on the brain of the subject. In some embodiments, the imaging technique is magnetic resonance imaging (MRI). In some embodiments, the imaging technique is positron emission tomography (PET).

In some embodiments, the neurological, muscular, or proliferative disorder is Parkinson's disease.

In some embodiments, treating or preventing the Parkinson's disease includes treating or preventing one or more symptoms of Parkinson's disease. In some embodiments, the one or more symptoms is selected from the group containing: tremors, stiffness of the arms, stiffness of the legs, stiffness of the trunk, slowness of movement, poor balance and coordination, and difficulty speaking.

In some embodiments, the symptom is tremors.

In some embodiments, the symptom is stiffness of the arms.

In some embodiments, the symptom is stiffness of the legs.

In some embodiments, the symptom is stiffness of the trunk.

In some embodiments, the symptom is slowness of movement.

In some embodiments, the symptom is poor balance and coordination.

In some embodiments, the symptom is difficulty speaking.

In some embodiments, the neurological, muscular, or proliferative disorder is vascular dementia. In some embodiments, treating or preventing the vascular dementia includes treating or preventing one or more symptoms of vascular dementia. In some embodiments, the one or more symptoms is selected from the group containing: short-term memory loss, getting lost in known surroundings, reduced concentration and planning, difficulty managing finances, difficulty following instructions, inability to control bladder, inability to control bowel, delusions, and hallucinations.

In some embodiments, the symptom is short-term memory loss.

In some embodiments, the symptom is getting lost in known surroundings.

In some embodiments, the symptom is reduced concentration and planning.

In some embodiments, the symptom is difficulty managing finances.

In some embodiments, the symptom is difficulty following instructions.

In some embodiments, the symptom is inability to control bladder.

In some embodiments, the symptom is inability to control bowel.

In some embodiments, the symptom is delusions.

In some embodiments, the symptom is hallucinations.

In some embodiments, the neurological, muscular, or proliferative disorder is dementia with Lewy bodies (DLB).

In some embodiments, the neurological, muscular, or proliferative disorder is dementia with Lewy bodies (DLB). In some embodiments, treating or preventing the dementia with Lewy bodies (DLB) includes treating or preventing one or more symptoms of dementia with Lewy bodies (DLB). In some embodiments, the one or more symptoms is selected from the group containing: visual hallucinations, rigid muscles, bradykinesia, shuffling walk or shaky legs, tremors, inability to balance the body, abnormally high or abnormally low blood pressure, sweating, digestion, confusion, poor concentration, memory loss, and difficulty sleeping.

In some embodiments, the symptom is visual hallucinations.

In some embodiments, the symptom is rigid muscles.

In some embodiments, the symptom is bradykinesia (i.e., slowed movement).

In some embodiments, the symptom is shuffling walk or shaky legs.

In some embodiments, the symptom is tremors.

In some embodiments, the symptom is inability to balance the body.

In some embodiments, the symptom is abnormally high or abnormally low blood pressure.

In some embodiments, the symptom is sweating.

In some embodiments, the symptom is digestion.

In some embodiments, the symptom is confusion.

In some embodiments, the symptom is poor concentration.

In some embodiments, the symptom is memory loss.

In some embodiments, the symptom is difficulty sleeping.

In some embodiments, the neurological, muscular, or proliferative disorder is Huntington disease (HD). In some embodiments, treating or preventing the Huntington disease (HD) includes treating or preventing one or more symptoms of Huntington disease (HD). In some embodiments, the one or more symptoms is selected from the group containing: chorea, tremors, muscle rigidity, slow or abnormal eye movements, impaired gait, impaired posture, impaired balance, difficulty swallowing, difficulty with speech, poor organizational aptitude, repetitive thoughts, repetitive behavior, repetitive actions, lack of impulse control, lack of social or behavioral awareness, inability to process thoughts, learning difficulties, difficulty selecting words to speak, dysgraphia, depression, mania, irritability, social withdrawal, insomnia, fatigue, suicidal thoughts or tendencies, and apathy.

In some embodiments, the symptom is chorea.

In some embodiments, the symptom is tremors.

In some embodiments, the symptom is muscle rigidity.

In some embodiments, the symptom is slow or abnormal eye movements.

In some embodiments, the symptom is impaired gait.

In some embodiments, the symptom is impaired posture.

In some embodiments, the symptom is impaired balance.

In some embodiments, the symptom is difficulty swallowing.

In some embodiments, the symptom is difficulty with speech.

In some embodiments, the symptom is poor organizational aptitude.

In some embodiments, the symptom is repetitive thoughts.

In some embodiments, the symptom is repetitive behavior.

In some embodiments, the symptom is repetitive actions.

In some embodiments, the symptom is lack of impulse control.

In some embodiments, the symptom is lack of social or behavioral awareness.

In some embodiments, the symptom is inability to process thoughts.

In some embodiments, the symptom is learning difficulties.

In some embodiments, the symptom is difficulty selecting words to speak.

In some embodiments, the symptom is dysgraphia.

In some embodiments, the symptom is depression.

In some embodiments, the symptom is mania.

In some embodiments, the symptom is irritability.

In some embodiments, the symptom is social withdrawal.

In some embodiments, the symptom is insomnia.

In some embodiments, the symptom is fatigue.

In some embodiments, the symptom is suicidal thoughts or tendencies.

In some embodiments, the symptom is apathy.

In some embodiments, the neurological, muscular, or proliferative disorder is amyotrophic lateral sclerosis (ALS). In some embodiments, treating or preventing the amyotrophic lateral sclerosis (ALS) includes treating or preventing one or more symptoms of amyotrophic lateral sclerosis (ALS). In some embodiments, the one or more symptoms is selected from the group containing: muscular weakness, difficulty walking, difficulty doing normal daily activities, clumsiness, slurred speech, difficulty swallowing, muscle cramps, twitching, poor posture, and falling.

In some embodiments, the symptom is muscular weakness.

In some embodiments, the symptom is difficulty walking.

In some embodiments, the symptom is difficulty doing normal daily activities.

In some embodiments, the symptom is clumsiness.

In some embodiments, the symptom is slurred speech.

In some embodiments, the symptom is difficulty swallowing.

In some embodiments, the symptom is muscle cramps.

In some embodiments, the symptom is twitching.

In some embodiments, the symptom is poor posture.

In some embodiments, the symptom is falling.

In some embodiments, the neurological, muscular, or proliferative disorder is Lafora disease. In some embodiments, treating or preventing the Lafora disease includes treating or preventing one or more symptoms of Lafora disease. In some embodiments, the one or more symptoms is selected from the group containing: seizures, myoclonus, dementia, headaches, and hallucinations (e.g., visual hallucinations).

In some embodiments, the symptom is seizures.

In some embodiments, the symptom is myoclonus.

In some embodiments, the symptom is dementia.

In some embodiments, the symptom is headaches.

In some embodiments, the symptom is hallucinations (e.g., visual hallucinations).

In some embodiments, the neurological, muscular, or proliferative disorder is neuroglioblastoma. In some embodiments, treating or preventing the neuroglioblastoma includes treating or preventing one or more symptoms of neuroglioblastoma. In some embodiments, the one or more symptoms is selected from the group containing: headaches, nausea, vomiting, blurred vision, double vision, and seizures.

In some embodiments, the symptom is headaches.

In some embodiments, the symptom is nausea.

In some embodiments, the symptom is vomiting.

In some embodiments, the symptom is blurred vision.

In some embodiments, the symptom is double vision.

In some embodiments, the symptom is seizures.

In some embodiments, the neurological, muscular, or proliferative disorder is diffuse intrinsic pontine glioma (DIPG). In some embodiments, treating or preventing the diffuse intrinsic pontine glioma (DIPG) includes treating or preventing one or more symptoms of diffuse intrinsic pontine glioma (DIPG). In some embodiments, the one or more symptoms is selected from the group containing: blurred vision, double vision, abnormal eye movement, and headaches.

In some embodiments, the symptom is blurred vision.

In some embodiments, the symptom is double vision.

In some embodiments, the symptom is abnormal eye movement.

In some embodiments, the symptom is headaches.

Some embodiments provide a method of increasing muscular strength in a subject identified or diagnosed as having muscular weakness, the method including administering a therapeutically effective amount of metformin or a pharmaceutically acceptable salt thereof and rapamycin or a pharmaceutically acceptable salt thereof to the subject.

Some embodiments provide a method of preventing or reversing muscle weakness in a subject in need thereof, the method including administering a therapeutically effective amount of metformin or a pharmaceutically acceptable salt thereof and rapamycin or a pharmaceutically acceptable salt thereof to the subject.

Some embodiments provide a method of preventing, slowing, or reversing sarcopenia in a subject in need thereof, the method including administering a therapeutically effective amount of metformin or a pharmaceutically acceptable salt thereof and rapamycin or a pharmaceutically acceptable salt thereof to the subject.

Disclosed herein is a method of treating or preventing myotonia in a subject in need thereof, the method including administering a therapeutically effective amount of metformin or a pharmaceutically acceptable salt thereof and rapamycin or a pharmaceutically acceptable salt thereof to the subject.

In some embodiments, the method includes determining an improvement in the stiffness visual analogue scale (VAS). In some embodiments, the method includes determining an improvement of at least 1 point (e.g., 1 point, 2 points, 3 points, or 4 points) in the stiffness visual analogue scale (VAS). Further information on the stiffness VAS can be found in Hammarén, Elisabet & Kjellby-Wendt, Gunilla & Lindberg, Christopher. (2005). Quantification of mobility impairment and self-assessment of stiffness in patients with myotonia congenita by the physiotherapist. Neuromuscular disorders, which is incorporated by reference herein in its entirety.

In some embodiments, the method includes determining a reduction in electrical activity in a muscle of a subject. In some embodiments, the method includes determining a reduction (e.g., at least 1% reduction, at least 2% reduction, at least 5% reduction, at least 7% reduction, at least 10% reduction, at least 15% reduction, at least 20% reduction, at least 30% reduction, or at least 40% reduction) in electrical activity in a muscle of a subject using electromyography (EMG).

Disclosed herein is a method of preventing muscle cell necrosis in a subject, including administering a therapeutically effective amount of metformin or a pharmaceutically acceptable salt thereof and rapamycin or a pharmaceutically acceptable salt thereof to the subject.

Disclosed herein is a method of improving balance and/or coordination in a subject, including administering a therapeutically effective amount of metformin or a pharmaceutically acceptable salt thereof and rapamycin or a pharmaceutically acceptable salt thereof to the subject.

In some embodiments, the method includes determining an improvement in the short physical performance battery (SPPB) after administering the metformin or a pharmaceutically acceptable salt thereof and rapamycin or a pharmaceutically acceptable salt thereof. In some embodiments, the method includes determining an improvement of at least 1 point (e.g., 2 points, 3 points, 4 points, 5 points, 6 points, 7 points, 8 points, 9 points, 10 points, or 11 points) in the short physical performance battery (SPPB) after administering the metformin or a pharmaceutically acceptable salt thereof and rapamycin or a pharmaceutically acceptable salt thereof. In some embodiments, determining an improvement in the short physical performance battery (SPPB) includes determining an improvement in the three-stage balance test. In some embodiments, determining an improvement in the short physical performance battery (SPPB) includes determining an improvement in the gait speed test. In some embodiments, determining an improvement in the short physical performance battery (SPPB) includes determining an improvement in the chair stand test. Further information on the SPPB can be found in, e.g., Cassidy B, Arena S. The Short Physical Performance Battery as a Predictor of Functional Decline. Home Healthcare Now. 2022 May 1; 40 (3): 168-9 which is incorporated by reference herein in its entirety.

In some embodiments, the method includes determining an increase in hand grip strength as measured by a dynamometer after administering the metformin or a pharmaceutically acceptable salt thereof and rapamycin or a pharmaceutically acceptable salt thereof. Further information can be found in, e.g., Journal of Bodywork and Movement Therapies, 2020, 24 (1), 235-243, which is incorporated by reference herein in its entirety.

In some embodiments, the method includes determining an at least 5% (e.g., at least 10% increase, at least 15% increase, at least 20% increase, at least 25% increase, at least 30% increase, at least 40% increase, at least 50% increase, at least 60% increase, at least 70% increase, at least 80% increase, at least 90% increase, or at least 95% increase) in hand grip strength as measured by a dynamometer after administering the metformin or a pharmaceutically acceptable salt thereof and rapamycin or a pharmaceutically acceptable salt thereof.

Some embodiments provide a method of increasing motility in a subject, the method including administering a therapeutically effective amount of metformin or a pharmaceutically acceptable salt thereof and rapamycin or a pharmaceutically acceptable salt thereof to the subject. In some embodiments, increasing motility in the subject includes increasing the number of voluntary muscle contractions in the subject per unit time (e.g., per minute, per 6 minutes, per 10 minutes, per 15 minutes, per 30 minutes, per 45 minutes, per hour, per 3 hours, per 12 hours, per 24 hours, per 2 days, per week, or per month) relative to before the therapeutically effective amount of metformin or a pharmaceutically acceptable salt thereof and rapamycin or a pharmaceutically acceptable salt thereof was administered to the subject. In some embodiments, increasing motility in the subject includes increasing the number of voluntary muscle contractions in the subject per minute. In some embodiments, increasing motility in the subject includes increasing the duration of a voluntary muscle contraction in the subject relative to before the therapeutically effective amount of metformin or a pharmaceutically acceptable salt thereof and rapamycin or a pharmaceutically acceptable salt thereof was administered to the subject. In some embodiments, increasing motility in the subject includes decreasing the length of time it takes for the subject to perform a task or an activity that requires movement and/or muscle contraction(s) relative to before administering the therapeutically effective amount of metformin or a pharmaceutically acceptable salt thereof and rapamycin or a pharmaceutically acceptable salt thereof was administered to the subject. In some embodiments, the method includes determining an improvement in the activity and social participation DM1-Active Scale after administering the metformin or a pharmaceutically acceptable salt thereof and rapamycin or a pharmaceutically acceptable salt thereof. Further information on the activity and social participation DM1-Active Scale can be found in, e.g., PLOS One 2015, 10: e0139944, which is incorporated by reference herein in its entirety.

In some embodiments, the method includes determining an improvement in the health-related quality of life InQoL questionnaire after administering the metformin or a pharmaceutically acceptable salt thereof and rapamycin or a pharmaceutically acceptable salt thereof. In some embodiments, determining an improvement in the health-related quality of life InQoL questionnaire includes determining an improvement in the fatigue subdomain. In some embodiments, determining an improvement in the health-related quality of life InQoL questionnaire includes determining an improvement in the activities subdomain. In some embodiments, determining an improvement in the health-related quality of life InQoL questionnaire includes determining an improvement in the independence subdomain. Further information on the health-related quality of life InQoL questionnaire can be found in, e.g., Taylor V R. Measuring healthy days; population assessment of health-related quality of life. Atlanta: US Centers for Disease Control and Prevention; 2000, which is incorporated by reference herein in its entirety.

In some embodiments, the method includes determining an improvement in at least one clinical parameter selected from the group consisting of: myotonia indices, muscle function and strength parameter, quality of life parameter, gait parameters, and total mechanical power of gait after administering the metformin or a pharmaceutically acceptable salt thereof and rapamycin or a pharmaceutically acceptable salt thereof. Further information on myotonia indices, muscle function and strength parameter, quality of life parameter, gait parameters, and total mechanical power of gait can be found in, e.g., Brain 2018:141, 2855-2865, which is incorporated by reference herein in its entirety.

In some embodiments, the method includes determining an increase in the distance walked by the subject during the 6 minute walk test (6MWT) after administering the metformin or a pharmaceutically acceptable salt thereof and rapamycin or a pharmaceutically acceptable salt thereof. In some embodiments, the method includes determining an at least 5% (e.g., at least 10%, at least 15%, at least 20%, at least 25%, at least 30%, at least 40%, at least 50%, at least 60%, at least 70%, at least 80%, at least 90%, at least 100%, at least 150%, at least 200%) increase in the distance walked by the subject during the 6 minute walk test (6MWT) after administering the metformin or a pharmaceutically acceptable salt thereof and rapamycin or a pharmaceutically acceptable salt thereof. Further information on the 6 minute walk test (6MWT) can be found in, e.g., Endocrinology 2005; 146:1328-1337, which is incorporated by reference herein in its entirety.

In some embodiments, the method includes determining a reduction in the time taken by the subject to complete the 10 meter walk test after administering the metformin or a pharmaceutically acceptable salt thereof and rapamycin or a pharmaceutically acceptable salt thereof. In some embodiments, the method includes determining an at least 5% (e.g., at least 10%, at least 15%, at least 20%, at least 25%, at least 30%, at least 40%, at least 50%, at least 60%, at least 70%, at least 80%, at least 90%) reduction in the time taken by the subject to complete the 10 meter walk test after administering the metformin or a pharmaceutically acceptable salt thereof and rapamycin or a pharmaceutically acceptable salt thereof. Further information on the 10 meter walk test can be found in, e.g., Journal of Neurologic Physical Therapy, 2018; 42 (2): 174-220, which is incorporated by reference herein in its entirety. In some embodiments, the method includes determining an improvement in at least one of the myotonia indices, muscle function and strength, quality of life, and total mechanical power after administering the metformin or a pharmaceutically acceptable salt thereof and rapamycin or a pharmaceutically acceptable salt thereof. In some embodiments, the method includes determining an at least 5% (e.g., at least 10%, at least 15%, at least 20%, at least 25%, at least 30%, at least 40%, at least 50%, at least 60%, at least 70%, at least 80%, at least 90%) reduction in the time taken by the subject to complete the 10 meter walk test after administering the metformin or a pharmaceutically acceptable salt thereof and rapamycin or a pharmaceutically acceptable salt thereof. Further information on the 10 meter walk test can be found in, e.g., Journal of Neurologic Physical Therapy, 2018; 42 (2): 174-220, which is incorporated by reference herein in its entirety.

In some embodiments, the method includes determining a reduction in the time taken by the subject to complete the 100 meter walk test after administering the metformin or a pharmaceutically acceptable salt thereof and rapamycin or a pharmaceutically acceptable salt thereof. In some embodiments, the method includes determining an at least 5% (e.g., at least 10%, at least 15%, at least 20%, at least 25%, at least 30%, at least 40%, at least 50%, at least 60%, at least 70%, at least 80%, at least 90%) reduction in the time taken by the subject to complete the 100 meter walk test after administering the metformin or a pharmaceutically acceptable salt thereof and rapamycin or a pharmaceutically acceptable salt thereof. Further information on the 100 meter walk test can be found in, e.g., Alfano, Lindsay & Miller, Natalie & Berry, Katherine & Yin, Han & Rolf, Kimberly & Flanigan, Kevin & Mendell, Jerry & Lowes, Linda. (2017). The 100-meter timed test: Normative data in healthy males and comparative pilot outcome data for use in Duchenne muscular dystrophy clinical trials. Neuromuscular Disorders. 27. 10.1016/j.nmd.2017.02.007, which is incorporated by reference herein in its entirety.

In some embodiments, the method includes determining an increase in muscle mass in the subject using magnetic resonance imaging (MRI) after administering the metformin or a pharmaceutically acceptable salt thereof and rapamycin or a pharmaceutically acceptable salt thereof. In some embodiments, the method includes determining an at least 1% increase (e.g., an at least 2% increase, an at least 3% increase, an at least 4% increase, an at least 5% increase, an at least 7% increase, an at least 9% increase, an at least 11% increase, an at least 13% increase, an at least 15% increase, an at least 20% increase, an at least 25% increase, an at least 30% increase, an at least 35% increase, or an at least 40% increase) in muscle mass in the subject using magnetic resonance imaging (MRI) after administering the metformin or a pharmaceutically acceptable salt thereof and rapamycin or a pharmaceutically acceptable salt thereof. In some embodiments, the muscle mass is skeletal muscle mass. In some embodiments, the increase in muscle mass is an increase in whole body muscle mass. Further information can be found in, e.g., Eur. J. Neurol. 2022 March; 29 (3): 843-854 or Radiography, 2015, 21 (1), e35-e39, each of which is incorporated by reference herein in its entirety.

In some embodiments, the method includes determining an improvement in the Muscular Impairment Rating Scale (MIRS) after administering the metformin or a pharmaceutically acceptable salt thereof and rapamycin or a pharmaceutically acceptable salt thereof. In some embodiments, an improvement in the MIRS includes an improvement in the scale of at least 1 point (e.g., 1 point, 2 points, 3 points, or 4 points). For further information on the MIRS, see, e.g., Neurology, 2001 Feb. 13; 56 (3): 336-40, which is incorporated by reference herein in its entirety.

In some embodiments, the method includes determining a decrease in the time measured for the subject to perform the timed up and go (TUG) test after administering the metformin or a pharmaceutically acceptable salt thereof and rapamycin or a pharmaceutically acceptable salt thereof. In some embodiments, the method includes determining an at least 2% decrease (e.g., an at least 3% decrease, an at least 4% decrease, an at least 5% decrease, an at least 7% decrease, an at least 9% decrease, an at least 11% decrease, an at least 13% decrease, an at least 15% decrease, an at least 20% decrease, an at least 25% decrease, an at least 30% decrease, an at least 35% decrease, an at least 40% decrease, an at least 50% decrease, an at least 60% decrease, or an at least 70% decrease) in the time measured for the subject to perform the timed up and go (TUG) test after administering the metformin or a pharmaceutically acceptable salt thereof and rapamycin or a pharmaceutically acceptable salt thereof. Further information on the timed up and go (TUG) test can be found in, e.g., Osteosarcopenia, 2022, pages 181-204, which is incorporated by reference herein in its entirety. Other tests that can be used to assess the mobility of a subject include, but are not limited to, the Pick up Weight Test (see, e.g., Reuben D B, Siu A L. “An objective measure of physical function of elderly outpatients. The Physical Performance Test”, J Am Geriatr Soc, 1990, vol. 38 (pg. 1105-12), incorporated by reference herein in its entirety), the Half Turn Test (see, e.g., Berg K, Wood-Dauphinee S, Williams J I, et al. Measuring balance in the elderly: preliminary development of an instrument, Physiother Can, 1989, vol. 41 (pg. 304-11), incorporated by reference herein in its entirety), the Alternate Step Test (see, e.g., Anne Tiedemann, Hiroyuki Shimada, Catherine Sherrington, Susan Murray, Stephen Lord, Age and Ageing, Volume 37, Issue 4, July 2008, Pages 430-435, incorporated by reference herein in its entirety), and the Stairs Ascent and Descent Test (see, e.g., Anne Tiedemann, Hiroyuki Shimada, Catherine Sherrington, Susan Murray, Stephen Lord, Age and Ageing, Volume 37, Issue 4, July 2008, Pages 430-435, incorporated by reference herein in its entirety).

Some embodiments provide a method of reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication in a subject in need thereof, the method including administering a therapeutically effective amount of metformin or a pharmaceutically acceptable salt thereof and rapamycin or a pharmaceutically acceptable salt thereof to the subject.

Some embodiments provide a method of preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication in a subject in need thereof, the method including administering a therapeutically effective amount of metformin or a pharmaceutically acceptable salt thereof and rapamycin or a pharmaceutically acceptable salt thereof to the subject.

In some embodiments, the method includes reducing or preventing cellular senescence. In some embodiments, the method includes preventing cellular senescence.

In some embodiments, the method includes reducing or preventing genomic instability.

In some embodiments, the method includes reducing or preventing telomere attrition.

In some embodiments, the method includes reducing or preventing epigenetic alterations (e.g., DNA methylation, histone modification, or regulation of the activity of genes by RNA and/or proteins).

In some embodiments, the method includes reducing or preventing dysregulation or deregulation of proteostasis.

In some embodiments, the method includes reducing or preventing deregulated nutrient sensing.

In some embodiments, the method includes reducing or preventing mitochondrial dysfunction.

In some embodiments, the method includes reducing or preventing stem cell exhaustion.

In some embodiments, the method includes reducing or preventing alteration of intercellular communication.

Some embodiments provide a method of promoting autophagy in a subject, the method including administering a therapeutically effective amount of metformin or a pharmaceutically acceptable salt thereof and rapamycin or a pharmaceutically acceptable salt thereof to the subject.

Some embodiments provide a method of darkening the color of the hair of a subject, the method including administering a therapeutically effective amount of metformin or a pharmaceutically acceptable salt thereof and rapamycin or a pharmaceutically acceptable salt thereof to the subject, where the color of the hair of the subject before administering the therapeutically effective amount of metformin or a pharmaceutically acceptable salt thereof and rapamycin or a pharmaceutically acceptable salt thereof to the subject is white or grey.

In some embodiments, the darkening includes changing the color of the hair to brown. In some embodiments, the darkening includes changing the color of the hair to black.

Some embodiments provide a method of improving memory in a subject in need thereof, the method including administering a therapeutically effective amount of metformin or a pharmaceutically acceptable salt thereof and rapamycin or a pharmaceutically acceptable salt thereof to the subject. In some embodiments, the improvement in memory in the subject is determined by the digit span test, the letter-number sequencing test, the California Verbal Learning Test, the Rey Auditory Verbal Learning Test, or the Wechsler Memory Scale. Further information on the digit span test, the letter-number sequencing test, the California Verbal Learning Test, the Rey Auditory Verbal Learning Test, and the Wechsler Memory Scale can be found at, e.g., Am Fam Physician. 2019, 99 (2), 101-108.

In some embodiments, the method comprises measuring an improvement in the digit span test in the subject after administering the therapeutically effective amount of metformin or a pharmaceutically acceptable salt thereof and rapamycin or a pharmaceutically acceptable salt thereof to the subject.

In some embodiments, the method comprises measuring an improvement in the letter-number sequencing test in the subject after administering the therapeutically effective amount of metformin or a pharmaceutically acceptable salt thereof and rapamycin or a pharmaceutically acceptable salt thereof to the subject.

In some embodiments, the method comprises measuring an improvement in the California Verbal Learning Test in the subject after administering the therapeutically effective amount of metformin or a pharmaceutically acceptable salt thereof and rapamycin or a pharmaceutically acceptable salt thereof to the subject.

In some embodiments, the method comprises measuring an improvement in the Rey Auditory Verbal Learning Test in the subject after administering the therapeutically effective amount of metformin or a pharmaceutically acceptable salt thereof and rapamycin or a pharmaceutically acceptable salt thereof to the subject.

In some embodiments, the method comprises measuring an improvement in the Wechsler Memory Scale in the subject after administering the therapeutically effective amount of metformin or a pharmaceutically acceptable salt thereof and rapamycin or a pharmaceutically acceptable salt thereof to the subject.

Some embodiments provide a method of preventing memory loss in a subject in need thereof, the method including administering a therapeutically effective amount of metformin or a pharmaceutically acceptable salt thereof and rapamycin or a pharmaceutically acceptable salt thereof to the subject.

Some embodiments provide a method of treating or preventing a neurological cancer in a subject in need thereof, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG)).

In some embodiments, the neurological cancer is neuroglioblastoma. In some embodiments, the neurological cancer is diffuse intrinsic pontine glioma (DIPG).

Some embodiments provide a method of inhibiting cell proliferation, the method including administering a therapeutically effective amount of metformin or a pharmaceutically acceptable salt thereof and rapamycin or a pharmaceutically acceptable salt thereof to the subject. In some embodiments, the cell is a mammalian cell. In some embodiments, the mammalian cell is a brain cell or spinal cell. In some embodiments, the brain cell or spinal cell is a glial cell, astrocyte, oligodendrocyte progenitor cell, neuron, or neural stem cell. In some embodiments, the inhibiting is performed in vivo. In some embodiments, the inhibiting is performed in vitro.

Some embodiments provide a method of treating a neurological cancer and/or inhibiting metastasis associated with a neurological cancer, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG)).

Some embodiments provide a method of providing supportive care to a cancer patient, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG).

Some embodiments provide a method for reversing or preventing acquired resistance to an anticancer drug in a subject who has a neurological cancer, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG)).

Some embodiments provide a method of treating a subject with a neurological cancer who has an increased likelihood of developing resistance to an anticancer drug, the method including administering a therapeutically effective amount of metformin or a pharmaceutically acceptable salt thereof and rapamycin or a pharmaceutically acceptable salt thereof to the subject; where the cancer is neuroglioblastoma or diffuse intrinsic pontine glioma (DIPG)).

In some embodiments, the subject has been identified or diagnosed as having a neurological cancer selected from neuroglioblastoma and diffuse intrinsic pontine glioma (DIPG).

In the field of medical oncology it is normal practice to use a combination of different forms of treatment to treat each patient with cancer. In medical oncology the other component(s) of such conjoint treatment or therapy in addition to the metformin or a pharmaceutically acceptable salt thereof and rapamycin or a pharmaceutically acceptable salt thereof may be, for example, surgery, radiotherapy, and chemotherapeutic agents, such as other kinase inhibitors, signal transduction inhibitors and/or monoclonal antibodies. For example, a surgery may be open surgery or minimally invasive surgery. The combination of metformin or a pharmaceutically acceptable salt thereof and rapamycin or a pharmaceutically acceptable salt thereof may also be useful as adjuvants to cancer treatment, that is, they can be used in combination with one or more additional therapies or therapeutic agents, for example a chemotherapeutic agent that works by the same or by a different mechanism of action.

In some embodiments of any the methods described herein, the metformin or a pharmaceutically acceptable salt thereof and rapamycin or a pharmaceutically acceptable salt thereof is administered in combination with a therapeutically effective amount of at least one additional therapeutic agent selected from one or more additional therapies or therapeutic (e.g., chemotherapeutic) agents. In some embodiments, the at least one additional therapeutic agent is selected from the group containing: alkylating agents (e.g., altretamine, busulfan, carboplatin, carmustine, cisplatin, cyclophosphamide, dacarbazine, ifosfamide, lomustine, melphalan, temozolomide, trabectedin), antimetabolites (e.g., 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, or a combination of trifluridine and tipiracil), alkaloids (e.g., vincristine, vinblastine, vinorelbine, paclitaxel, docetaxel, etoposide, teniposide, irinotecan, topotecan), and anti-tumor antibiotics (e.g., daunorubicin, doxorubicin, doxorubicin liposomal, epirubicin, idarubicin, valrubicin). Some therapeutic agents useful for treating neuroglioblastoma include, but are not limited to, cyclophosphamide, danyelza (naxitamab), dinutuximab, doxorubicin, naxitamab, unituxin (dinutuximab), vincristine sulfate, busulfan, and melphalan. Some therapies useful for treating diffuse intrinsic pontine glioma (DIPG) include, but are not limited to the following: fractionated radiation therapy with concomitant administration of anti-inflammatory steroids (e.g., dexamethasone), anti-GD2 CAR T cells, and anti-EGFR drugs (e.g., nimotuzumab, gefitinib, and erlotinib). In some embodiments, the at least one additional therapeutic agent is selected from the group containing: alkylating agents (e.g., altretamine, busulfan, carboplatin, carmustine, cisplatin, cyclophosphamide, dacarbazine, ifosfamide, lomustine, melphalan, temozolomide, trabectedin), antimetabolites (e.g., 5-fluorouracil, 6-mercaptopurine, azacitidine, capecitabine, clofarabine, cytarabine, floxuridine, fludarabine, gemcitabine, methotrexate, pemetrexed, pentostatin, pralatrexate, or a combination of trifluridine and tipiracil), alkaloids (e.g., vincristine, vinblastine, vinorelbine, paclitaxel, docetaxel, etoposide, teniposide, irinotecan, topotecan), and anti-tumor antibiotics (e.g., daunorubicin, doxorubicin, doxorubicin liposomal, epirubicin, idarubicin, valrubicin), cyclophosphamide, danyelza (naxitamab), dinutuximab, doxorubicin, naxitamab, unituxin (dinutuximab), vincristine sulfate, busulfan, melphalan, anti-GD2 CAR T cells, and anti-EGFR drugs (e.g., nimotuzumab, gefitinib, and erlotinib).

Accordingly, also provided herein is a method of treating a neurological cancer, including administering to a patient in need thereof a pharmaceutical combination for treating cancer which includes (a) metformin or a pharmaceutically acceptable salt thereof and rapamycin or a pharmaceutically acceptable salt thereof, (b) an additional therapeutic agent, and (c) optionally at least one pharmaceutically acceptable carrier for simultaneous, separate, or sequential use for the treatment of cancer, where the amounts of the compound of metformin or a pharmaceutically acceptable salt thereof and rapamycin or a pharmaceutically acceptable salt thereof and the additional therapeutic agent are together effective in treating the cancer; where the cancer is neuroglioblastoma or diffuse intrinsic pontine glioma (DIPG)).

Some embodiments provide a method for inhibiting, preventing, aiding in the prevention, or decreasing the symptoms of metastasis of a neurological cancer in a patient in need thereof, the method including administering to the patient a therapeutically effective amount of metformin or a pharmaceutically acceptable salt thereof and rapamycin or a pharmaceutically acceptable salt thereof; where the cancer is neuroglioblastoma or diffuse intrinsic pontine glioma (DIPG)). The term “metastasis” is an art known term and means the formation of an additional tumor (e.g., a solid tumor) at a site distant from a primary tumor in a subject or patient, where the additional tumor includes the same or similar cancer cells as the primary tumor.

In some embodiments, after administering the metformin or a pharmaceutically acceptable salt thereof and rapamycin or a pharmaceutically acceptable salt thereof, an increase in AMPK activation, inhibition of mTORC1, inhibition of S6 kinase, or any combination thereof are determined in the subject. In some embodiments, after administering the metformin or a pharmaceutically acceptable salt thereof and rapamycin or a pharmaceutically acceptable salt thereof, an increase in AMPK activation, inhibition of mTORC1, and inhibition of S6 kinase are determined in the subject.

In some embodiments, after administering the metformin or a pharmaceutically acceptable salt thereof and rapamycin or a pharmaceutically acceptable salt thereof, p70 S6 kinase is inhibited or abolished in the subject.

In some embodiments, after administering the metformin or a pharmaceutically acceptable salt thereof and rapamycin or a pharmaceutically acceptable salt thereof, Akt is activated in the subject.

In some embodiments, the metformin or a pharmaceutically acceptable salt thereof is metformin hydrochloride.

In some embodiments, the rapamycin or a pharmaceutically acceptable salt thereof is rapamycin.

In some embodiments, a cyclosporine, tacrolimus, and mycophenolate mofetil were not previously administered to the subject within 1 month of administering the metformin or a pharmaceutically acceptable salt thereof and rapamycin or a pharmaceutically acceptable salt thereof.

In some embodiments, the subject is not identified or diagnosed as having a disease associated with a kidney.

In some embodiments, the subject is not identified or diagnosed as having a disease associated with the liver.

In some embodiments, the subject is not identified or diagnosed as having a disease associated with the heart.

In some embodiments, the subject is not identified or diagnosed as having diabetes.

In some embodiments, the subject is not identified or diagnosed as having abnormal endocrine function.

In some embodiments, the subject was not previously administered a therapeutic agent that modulates the insulin transduction pathway within 1 year of administering the metformin or a pharmaceutically acceptable salt thereof and rapamycin or a pharmaceutically acceptable salt thereof.

The amount administered depends on the compound formulation, route of administration, the disorder treated or prevented, the therapeutic outcome sought, etc. and is generally empirically determined, and variations will necessarily occur depending on the target, the host, and the route of administration, etc. Generally, the quantity of active compound in a unit dose of preparation may be varied or adjusted, according to the particular application. For convenience, the total daily dosage may be divided and administered in portions during the day.

In some embodiments, the metformin or a pharmaceutically acceptable salt thereof is administered monthly, every 3 weeks, every 2 weeks, every 10 days, every 9 days, every 8 days, every 7 days, every 6 days, every 5 days, every 4 days, every 3 days, every 2 days, every day (i.e., daily), 3 times per week, 2 times per week, twice per day, or three times per day. In some embodiments, the metformin or a pharmaceutically acceptable salt thereof is administered daily. In some embodiments, an initial dose of metformin (i.e., a “loading dose”) is administered to the subject as the first dose of the treatment. In some embodiments, the initial dose of metformin is higher than subsequent doses administered to the subject.

In some embodiments, each dose of metformin or a pharmaceutically acceptable salt thereof is from about 100 mg to about 5000 mg (e.g., about 100 mg to about 500 mg, about 100 mg to about 800 mg, about 100 mg to about 1000 mg, about 500 mg to about 1000 mg, about 1000 mg to about 1500 mg, about 1500 mg to about 2250 mg about 1500 mg to about 2000 mg, about 2000 mg to about 2500 mg, about 2250 mg to about 3000 mg, about 2500 mg to about 3000 mg, about 3000 mg to about 3500 mg, about 3500 mg to about 4000 mg, about 4000 mg to about 5000 mg, about 500 mg to about 3000 mg, about 500 to about 1750 mg, about 500 mg to about 1125 mg, about 1125 mg to about 2250 mg, about 1500 mg to about 3000 mg, about 500 mg, about 650 mg, about 750 mg, about 850 mg, about 1000 mg, about 1750 mg, about 2000 mg, or about 3000 mg) on a free base basis of metformin. In some embodiments, the dose of metformin or a pharmaceutically acceptable salt thereof is from about 500 to about 3000 mg on a free base basis of metformin. In some embodiments, the dose of metformin or a pharmaceutically acceptable salt thereof is from about 1500 to about 3000 mg on a free base basis of metformin. In some embodiments, the dose of metformin or a pharmaceutically acceptable salt thereof is from about 500 to about 1750 mg on a free base basis of metformin.

In some embodiments, the method includes treating or preventing myotonic dystrophy type 1 (DM1); and the metformin or a pharmaceutically acceptable salt thereof is administered in a daily dose of about 1500 mg to about 3000 mg on a free base basis of metformin.

In some embodiments, the method includes treating or preventing Duchenne muscular dystrophy; and the metformin or a pharmaceutically acceptable salt thereof is administered in a daily dose of about 1500 mg to about 3000 mg on a free base basis of metformin.

In some embodiments, the method includes treating or preventing spinocerebellar ataxia type 3 (SCA-3); and the metformin or a pharmaceutically acceptable salt thereof is administered in a daily dose of about 1500 mg to about 3000 mg on a free base basis of metformin.

In some embodiments, the method includes treating or preventing Alzheimer's disease; and the metformin or a pharmaceutically acceptable salt thereof is administered in a daily dose of about 500 mg to about 1750 mg on a free base basis of metformin.

In some embodiments, the method includes promoting autophagy; and the metformin or a pharmaceutically acceptable salt thereof is administered in a daily dose of about 500 mg to about 1750 mg on a free base basis of metformin.

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; and the metformin or a pharmaceutically acceptable salt thereof is administered in a daily dose of about 500 mg to about 1750 mg on a free base basis of metformin.

In some embodiments, the rapamycin or a pharmaceutically acceptable salt thereof is administered monthly, every 3 weeks, every 2 weeks, every 10 days, every 9 days, every 8 days, every 7 days, every 6 days, every 5 days, every 4 days, every 3 days, every 2 days, every day (i.e., daily), 3 times per week, 2 times per week, twice per day, or three times per day. In some embodiments, the rapamycin or a pharmaceutically acceptable salt thereof is administered daily. In some embodiments, the rapamycin or a pharmaceutically acceptable salt thereof is administered weekly. In some embodiments, the rapamycin or a pharmaceutically acceptable salt thereof is administered on a daily basis in addition to another dose on a weekly basis. In some embodiments, an initial dose of rapamycin (i.e., a “loading dose”) is administered to the subject as the first dose of the treatment. In some embodiments, the initial dose of rapamycin is higher than subsequent doses administered to the subject. In some embodiments, the dose of rapamycin or a pharmaceutically acceptable salt thereof is from about 0.1 mg to about 25 mg (e.g., about 0.1 mg to about 20 mg, about 0.1 mg to about 18 mg, about 0.1 mg to about 15 mg, about 0.1 mg to about 13 mg, about 0.1 mg to about 10 mg, about 0.1 mg to about 7 mg, about 0.1 mg to about 5 mg, about 0.1 mg to about 3 mg, about 0.1 mg to about 2 mg, about 0.1 mg to about 1 mg, about 0.5 mg to about 20 mg, about 0.5 mg to about 18 mg, about 0.5 mg to about 15 mg, about 0.5 mg to about 13 mg, about 0.5 mg to about 10 mg, about 0.5 mg to about 7 mg, about 0.5 mg to about 5 mg, about 0.5 mg to about 3 mg, about 0.5 mg to about 2 mg, about 0.5 mg to about 1 mg, about 1 mg to about 20 mg, about 1 mg to about 18 mg, about 1 mg to about 15 mg, about 1 mg to about 13 mg, about 1 mg to about 10 mg, about 1 mg to about 7 mg, about 1 mg to about 5 mg, about 1 mg to about 3 mg, about 1 mg to about 2 mg, about 2 mg to about 12 mg, about 4 mg to about 10 mg, about 4 mg to about 8 mg, about 10 mg to about 30 mg, about 13 mg to about 17 mg, about 2 mg to about 4 mg, about 2 mg to about 10 mg, about 1 mg to about 3 mg, about 3 mg to about 7 mg, about 4 mg to about 6 mg, about 15 mg to about 25 mg, about 18 mg to about 22 mg, about 10 mg to about 14 mg, about 0.5 mg, about 1 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg) on a free base basis of rapamycin. In some embodiments, the dose of rapamycin or a pharmaceutically acceptable salt thereof is from about 0.5 mg to about 1 mg on a free base basis of rapamycin. In some embodiments, when the subject is 12 years or younger, the dose is about 50% of the dose administered to subjects greater than 12 years old. In some embodiments, the dose of rapamycin or a pharmaceutically acceptable salt thereof is about 0.7 mg on a free base basis of rapamycin. In some embodiments, the dose of rapamycin or a pharmaceutically acceptable salt thereof is from about 1 mg to about 10 mg on a free base basis of rapamycin. In some embodiments, the dose of rapamycin or a pharmaceutically acceptable salt thereof is from about 2 mg to about 10 mg on a free base basis of rapamycin. In some embodiments, the dose of rapamycin or a pharmaceutically acceptable salt thereof is about 5 mg on a free base basis of rapamycin.

In some embodiments, the rapamycin or a pharmaceutically acceptable salt thereof is administered as a daily dose of about 0.1 mg to about 2 mg on a free base basis of rapamycin; and an additional weekly dose of about 1 mg to about 10 mg on a free base basis of rapamycin.

In some embodiments, the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin.

In some embodiments, the rapamycin or a pharmaceutically acceptable salt thereof is administered in a daily dose of about 0.7 mg on a free base basis of rapamycin; and an additional weekly dose of about 5 mg on a free base basis of rapamycin.

In some embodiments, the method includes treating or preventing myotonic dystrophy type 1 (DM1); and the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin.

In some embodiments, the method includes treating or preventing myotonic dystrophy type 1 (DM1); and the rapamycin or a pharmaceutically acceptable salt thereof is administered as a daily dose of about 0.5 mg to about 1 mg on a free base basis of rapamycin.

In some embodiments, the method includes treating or preventing myotonic dystrophy type 1 (DM1); and the rapamycin or a pharmaceutically acceptable salt thereof is administered as a weekly dose of about 2 mg to about 10 mg on a free base basis of rapamycin.

In some embodiments, the method includes treating or preventing Duchenne muscular dystrophy; and the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin.

In some embodiments, the method includes treating or preventing Duchenne muscular dystrophy; and the rapamycin or a pharmaceutically acceptable salt thereof is administered as a daily dose of about 0.5 mg to about 1 mg on a free base basis of rapamycin.

In some embodiments, the method includes treating or preventing Duchenne muscular dystrophy; and the rapamycin or a pharmaceutically acceptable salt thereof is administered as a weekly dose of about 2 mg to about 10 mg on a free base basis of rapamycin.

In some embodiments, the method includes treating or preventing spinocerebellar ataxia type 3 (SCA-3); and the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin.

In some embodiments, the method includes treating or preventing spinocerebellar ataxia type 3 (SCA-3); and the rapamycin or a pharmaceutically acceptable salt thereof is administered as a daily dose of about 0.5 mg to about 1 mg on a free base basis of rapamycin.

In some embodiments, the method includes treating or preventing spinocerebellar ataxia type 3 (SCA-3); and the rapamycin or a pharmaceutically acceptable salt thereof is administered as a weekly dose of about 2 mg to about 10 mg on a free base basis of rapamycin.

In some embodiments, the method includes treating or preventing Alzheimer's disease; and the rapamycin or a pharmaceutically acceptable salt thereof is administered as a daily dose of about 0.5 mg to about 1 mg on a free base basis of rapamycin; and an additional weekly dose of about 1 mg to about 10 mg on a free base basis of rapamycin.

In some embodiments, the method includes treating or preventing Alzheimer's disease; and the rapamycin or a pharmaceutically acceptable salt thereof is administered as a daily dose of about 0.5 mg to about 1 mg on a free base basis of rapamycin.

In some embodiments, the method includes treating or preventing Alzheimer's disease; and the rapamycin or a pharmaceutically acceptable salt thereof is administered as a weekly dose of about 1 mg to about 10 mg on a free base basis of rapamycin.

In some embodiments, the method includes promoting autophagy; and the rapamycin or a pharmaceutically acceptable salt thereof is administered as a daily dose of about 0.5 mg to about 1 mg on a free base basis of rapamycin; and an additional weekly dose of about 1 mg to about 10 mg on a free base basis of rapamycin.

In some embodiments, the method includes promoting autophagy; and the rapamycin or a pharmaceutically acceptable salt thereof is administered as a daily dose of about 0.5 mg to about 1 mg on a free base basis of rapamycin.

In some embodiments, the method includes promoting autophagy; and the rapamycin or a pharmaceutically acceptable salt thereof is administered as a weekly dose of about 1 mg to about 10 mg on a free base basis of rapamycin.

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the rapamycin or a pharmaceutically acceptable salt thereof is administered as a daily dose of about 0.5 mg to about 1 mg on a free base basis of rapamycin; and an additional weekly dose of about 1 mg to about 10 mg on a free base basis of rapamycin.

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the rapamycin or a pharmaceutically acceptable salt thereof is administered as a daily dose of about 0.5 mg to about 1 mg on a free base basis of rapamycin.

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the rapamycin or a pharmaceutically acceptable salt thereof is administered as a weekly dose of about 1 mg to about 10 mg on a free base basis of rapamycin.

In some embodiments, the metformin or a pharmaceutically acceptable salt thereof is administered in a daily dose of about 1500 mg to about 3000 mg; and the rapamycin or a pharmaceutically acceptable salt thereof is administered as a daily dose of about 0.5 mg to about 1 mg on a free base basis of rapamycin.

In some embodiments, the metformin or a pharmaceutically acceptable salt thereof is administered in a daily dose of about 1500 mg to about 3000 mg; and the rapamycin or a pharmaceutically acceptable salt thereof is administered as a weekly dose of about 2 mg to about 10 mg on a free base basis of rapamycin.

In some embodiments, the metformin or a pharmaceutically acceptable salt thereof is administered in a daily dose of about 1500 mg to about 3000 mg; and the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin.

In some embodiments, the metformin or a pharmaceutically acceptable salt thereof is administered in a daily dose of about 1500 mg to about 3000 mg; and the rapamycin or a pharmaceutically acceptable salt thereof is administered as a daily dose of about 0.5 mg to about 1 mg on a free base basis of rapamycin; and an additional weekly dose of about 2 mg to about 10 mg on a free base basis of rapamycin; where the daily dose of metformin or a pharmaceutically acceptable salt thereof and the daily dose of rapamycin or a pharmaceutically acceptable salt thereof are administered simultaneously (e.g., as a fixed dosage form).

In some embodiments, the metformin or a pharmaceutically acceptable salt thereof is administered in a daily dose of about 500 mg to about 1750 mg; and the rapamycin or a pharmaceutically acceptable salt thereof is administered as a daily dose of about 0.5 mg to about 1 mg on a free base basis of rapamycin; and an additional weekly dose of about 1 mg to about 10 mg on a free base basis of rapamycin.

In some embodiments, the metformin or a pharmaceutically acceptable salt thereof is administered in a daily dose of about 500 mg to about 1750 mg; and the rapamycin or a pharmaceutically acceptable salt thereof is administered as a daily dose of about 0.5 mg to about 1 mg on a free base basis of rapamycin; and an additional weekly dose of about 1 mg to about 10 mg on a free base basis of rapamycin; where the daily dose of metformin or a pharmaceutically acceptable salt thereof and the daily dose of rapamycin or a pharmaceutically acceptable salt thereof are administered simultaneously (e.g., as a fixed dosage form).

In some embodiments, when the metformin or a pharmaceutically acceptable salt thereof is administered on the same day as the rapamycin or a pharmaceutically acceptable salt thereof, both are administered simultaneously (e.g., as a fixed dosage form (e.g., as an oral fixed dosage form)). In some embodiments, when metformin or a pharmaceutically acceptable salt thereof and rapamycin or a pharmaceutically acceptable salt thereof are each administered daily, they are administered together as a fixed dosage form. In some embodiments, when metformin or a pharmaceutically acceptable salt thereof is administered daily and rapamycin or a pharmaceutically acceptable salt thereof is administered weekly, the weekly dose of rapamycin or a pharmaceutically acceptable salt thereof is administered as a fixed dosage form with the daily dose of metformin or a pharmaceutically acceptable salt thereof that is administered on the same day as the weekly dose of rapamycin or a pharmaceutically acceptable salt thereof.

In some embodiments, the method includes treating or preventing myotonic dystrophy type 1 (DM1); the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 2 mg to about 10 mg on a free base basis of rapamycin.

In some embodiments, the method includes treating or preventing myotonic dystrophy type 1 (DM1); the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes treating or preventing myotonic dystrophy type 1 (DM1); the metformin or a pharmaceutically acceptable salt thereof is administered in a daily dose of about 1500 mg to about 3000 mg on a free base basis of metformin; and the rapamycin or a pharmaceutically acceptable salt thereof is administered as a weekly dose of about 2 mg to about 10 mg on a free base basis of rapamycin (e.g., as a fixed dosage form with the metformin or a pharmaceutically acceptable salt thereof).

In some embodiments, the method includes treating or preventing Duchenne muscular dystrophy; the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 2 mg to about 10 mg on a free base basis of rapamycin.

In some embodiments, the method includes treating or preventing Duchenne muscular dystrophy; the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes treating or preventing Duchenne muscular dystrophy; the metformin or a pharmaceutically acceptable salt thereof is administered in a daily dose of about 1500 mg to about 3000 mg on a free base basis of metformin; and the rapamycin or a pharmaceutically acceptable salt thereof is administered as a weekly dose of about 2 mg to about 10 mg on a free base basis of rapamycin (e.g., as a fixed dosage form with the metformin or a pharmaceutically acceptable salt thereof).

In some embodiments, the method includes treating or preventing spinocerebellar ataxia type 3 (SCA-3); the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 2 mg to about 10 mg on a free base basis of rapamycin.

In some embodiments, the method includes treating or preventing spinocerebellar ataxia type 3 (SCA-3); the metformin or a pharmaceutically acceptable salt thereof is administered in a daily dose of about 1500 mg to about 3000 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes treating or preventing spinocerebellar ataxia type 3 (SCA-3); the metformin or a pharmaceutically acceptable salt thereof is administered in a daily dose of about 1500 mg to about 3000 mg on a free base basis of metformin; and the rapamycin or a pharmaceutically acceptable salt thereof is administered as a weekly dose of about 2 mg to about 10 mg on a free base basis of rapamycin (e.g., as a fixed dosage form with the metformin or a pharmaceutically acceptable salt thereof).

In some embodiments, the method includes treating or preventing Alzheimer's disease; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 1 mg to about 10 mg on a free base basis of rapamycin.

In some embodiments, the method includes treating or preventing Alzheimer's disease; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes treating or preventing Alzheimer's disease; the metformin or a pharmaceutically acceptable salt thereof is administered in a daily dose of about 500 mg to about 1750 mg on a free base basis of metformin; and the rapamycin or a pharmaceutically acceptable salt thereof is administered as a weekly dose of about 1 mg to about 10 mg on a free base basis of rapamycin (e.g., as a fixed dosage form with the metformin or a pharmaceutically acceptable salt thereof).

In some embodiments, the method includes promoting autophagy; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 1 mg to about 10 mg on a free base basis of rapamycin.

In some embodiments, the method includes promoting autophagy; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes promoting autophagy; the metformin or a pharmaceutically acceptable salt thereof is administered in a daily dose of about 500 mg to about 1750 mg on a free base basis of metformin; and the rapamycin or a pharmaceutically acceptable salt thereof is administered as a weekly dose of about 1 mg to about 10 mg on a free base basis of rapamycin (e.g., as a fixed dosage form with the metformin or a pharmaceutically acceptable salt thereof).

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof); and an additional weekly dose of about 1 mg to about 10 mg on a free base basis of rapamycin.

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the metformin or a pharmaceutically acceptable salt thereof is administered in a daily dose of about 500 mg to about 1750 mg on a free base basis of metformin; and the rapamycin or a pharmaceutically acceptable salt thereof is administered as a daily dose of about 0.5 mg to about 1 mg on a free base basis of rapamycin (e.g., as a fixed dosage form with the metformin or a pharmaceutically acceptable salt thereof).

In some embodiments, the method includes reducing or preventing genomic instability, telomere attrition, epigenetic alterations (e.g., DNA methylation, histone modification, or regulation of the activity of genes by RNA and/or proteins), dysregulation or deregulation of proteostasis, deregulated nutrient sensing, mitochondrial dysfunction, cellular senescence, stem cell exhaustion, and/or alteration of intercellular communication; the metformin or a pharmaceutically acceptable salt thereof is administered in a daily dose of about 500 mg to about 1750 mg on a free base basis of metformin; and the rapamycin or a pharmaceutically acceptable salt thereof is administered as a weekly dose of about 1 mg to about 10 mg on a free base basis of rapamycin (e.g., as a fixed dosage form with the metformin or a pharmaceutically acceptable salt thereof).

In some embodiments, the metformin or a pharmaceutically acceptable salt thereof and the rapamycin or a pharmaceutically acceptable salt thereof are administered separately, sequentially, or simultaneously. In some embodiments, the metformin or a pharmaceutically acceptable salt thereof and the rapamycin or a pharmaceutically acceptable salt thereof are administered separately. In some embodiments, the metformin or a pharmaceutically acceptable salt thereof and the rapamycin or a pharmaceutically acceptable salt thereof are administered sequentially. In some embodiments, the metformin or a pharmaceutically acceptable salt thereof and the rapamycin or a pharmaceutically acceptable salt thereof are administered simultaneously. In some embodiments, the metformin or a pharmaceutically acceptable salt thereof and the rapamycin or a pharmaceutically acceptable salt thereof are administered simultaneously as a fixed dosage form.

In some embodiments, the period of administration of the metformin or a pharmaceutically acceptable salt thereof and the rapamycin or a pharmaceutically acceptable salt thereof is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, about 5 days to about 14 days, about 1 day to about 1 month, about 1 day, to about two weeks, at least about 1 month, 4 months, 5 months, 6 months, 7 months, 8 months, 9 months, 10 months, 11 months, 12 months, at least about 1 year, at least about 2 years, at least about 5 years, at least about 10 years, at least about 15 years, at least about 20 years, or longer. In some embodiments, the period of administration of the metformin or a pharmaceutically acceptable salt thereof and the rapamycin or a pharmaceutically acceptable salt thereof is from about 1 day to about 1 month. In some embodiments, the period of administration of the metformin or a pharmaceutically acceptable salt thereof and the rapamycin or a pharmaceutically acceptable salt thereof is from about 1 day to about two weeks. In some embodiments, the period of administration of the metformin or a pharmaceutically acceptable salt thereof and the rapamycin or a pharmaceutically acceptable salt thereof is about two weeks. In some embodiments, the period of administration of the metformin or a pharmaceutically acceptable salt thereof and the rapamycin or a pharmaceutically acceptable salt thereof is about 12 days. In some embodiments, the period of administration of the metformin or a pharmaceutically acceptable salt thereof and the rapamycin or a pharmaceutically acceptable salt thereof is about one week. In some embodiments, the period of administration of the metformin or a pharmaceutically acceptable salt thereof and the rapamycin or a pharmaceutically acceptable salt thereof is for at least about one month. In some embodiments, the period of administration of the metformin or a pharmaceutically acceptable salt thereof and the rapamycin or a pharmaceutically acceptable salt thereof is for at least about one year. In some embodiments, a period during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or greater than 12 months. In some embodiments, a period during which administration is stopped is for up to 1 day, up to 2 days, up to 3 days, up to 4 days, up to 5 days, up to 6 days, up to 7 days, up to 8 days, up to 9 days, up to 10 days, up to 11 days, up to 12 days, up to 13 days, up to 14 days, up to 3 weeks, up to 4 weeks, up to 5 weeks, up to 6 weeks, up to 7 weeks, up to 8 weeks, up to 9 weeks, up to 10 weeks, up to 11 weeks, up to 12 weeks, up to 4 months, up to 5 months, up to 6 months, up to 7 months, up to 8 months, up to 9 months, up to 10 months, up to 11 months, or up to 12 months. In an embodiment, the metformin or a pharmaceutically acceptable salt thereof and the rapamycin or a pharmaceutically acceptable salt thereof is administered to an individual for a period of time followed by a separate period of time. In another embodiment, the metformin or a pharmaceutically acceptable salt thereof and the rapamycin or a pharmaceutically acceptable salt thereof is administered for a first period and a second period following the first period, with administration stopped during the second period, followed by a third period where administration of the metformin or a pharmaceutically acceptable salt thereof and the rapamycin or a pharmaceutically acceptable salt thereof is started and then a fourth period following the third period where administration is stopped. In an aspect of this embodiment, the period of administration of the metformin or a pharmaceutically acceptable salt thereof and the rapamycin or a pharmaceutically acceptable salt thereof followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or greater. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, after administration (for example, after oral administration) of the metformin or a pharmaceutically acceptable salt thereof and rapamycin or a pharmaceutically acceptable salt thereof, the subject experiences gastrointestinal symptoms that are ameliorated by the consumption of food prior to administration of the metformin or a pharmaceutically acceptable salt thereof and rapamycin or a pharmaceutically acceptable salt thereof. In some embodiments, the subject consumes food up to about 6 hours before administering the metformin or a pharmaceutically acceptable salt thereof and rapamycin or a pharmaceutically acceptable salt thereof. For example, the subject consumes food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the metformin or a pharmaceutically acceptable salt thereof and rapamycin or a pharmaceutically acceptable salt thereof. For example, the subject consumes food concurrently with administering the metformin or a pharmaceutically acceptable salt thereof and rapamycin or a pharmaceutically acceptable salt thereof.

In some embodiments, the subject is less than 90 years of age, e.g., less than 80 years of age, less than 70 years of age, less than 60 years of age, less than 50 years of age, less than 40 years of age, less than 30 years of age, less than 15 years of age, less than 10 years of age, less than 5 years of age, about 1 week to about 5 years of age, about 5 years of age to about 12 years of age, about 12 years of age to about 21 years of age, about 21 years of age to about 34 years of age, about 34 years of age to about 45 years of age, about 45 years of age to about 55 years of age, about 55 years of age to about 65 years of age, about 65 years of age to about 75 years of age, or about 75 years of age to about 90 years of age. In some embodiments, the subject is less than 80 years of age. In some embodiments, the subject is less than 70 years of age. In some embodiments, the subject is less than 60 years of age.

Solid dosage forms of the instant pharmaceutical compositions for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents.

Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the instant pharmaceutical compositions of tablets, dragées, capsules, pills, and granules can be prepared with coatings and shells such as enteric coatings and other pharmaceutical coatings. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Liquid dosage forms of the instant pharmaceutical compositions for oral administration include pharmaceutically acceptable emulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Suspensions of the instant compounds, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Pharmaceutical compositions of the present disclosure for injection include pharmaceutically acceptable sterile aqueous or non-aqueous solutions, dispersions, suspensions, or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

Besides inert diluents, these pharmaceutical compositions may also contain adjuvants such as preservative, wetting agents, emulsifying agents, dispersing agents, sweetening, flavoring, and perfuming agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin. The compounds can be incorporated into slow release or targeted delivery systems such as polymer matrices, liposomes, and microspheres. Such formulations may provide more effective distribution of the compounds.

The pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable medium prior to use.

Dosage forms for topical administration of a compound or pharmaceutical composition of the present disclosure include powders, patches, sprays, ointments, and inhalants. The active compound is mixed under sterile conditions with a pharmaceutically acceptable carrier and any preservatives, buffers, or propellants which may be required.

The compounds and compositions described herein can, for example, be administered orally, parenterally (e.g., subcutaneously, intracutaneously, intravenously or intramuscularly), topically, rectally, nasally sublingually or buccally, with a dosage ranging from about 0.01 milligrams per kilogram (mg/kg) to about 1000 mg/kg, (e.g., from about 0.01 to about 100 mg/kg, from about 0.1 to about 100 mg/kg) every 4 to 120 hours, or according to the requirements of the particular drug, dosage form, and/or route of administration. Other routes of administration include enteric, intraarterial, intraperitoneal and intrathecal administration. The interrelationship of dosages for animals and humans (based on milligrams per meter squared of body surface) is described by Freireich et al., Cancer Chemother. Rep. 50, 219-244 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or by injection. The methods herein contemplate administration of a therapeutically effective amount of compound or compound composition to achieve a desired or stated effect. Typically, the pharmaceutical compositions of the present disclosure will be administered from about 1 to about 6 times per day or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, and the judgment of the treating physician.

Provided in the present disclosure are dosage forms including metformin or a pharmaceutically acceptable salt thereof in an amount of from about 100 mg to about 5000 mg (e.g., about 100 mg to about 500 mg, about 100 mg to about 800 mg, about 100 mg to about 1000 mg, about 500 mg to about 1000 mg, about 1000 mg to about 1500 mg, about 1500 mg to about 2250 mg about 1500 mg to about 2000 mg, about 2000 mg to about 2500 mg, about 2250 mg to about 3000 mg, about 2500 mg to about 3000 mg, about 3000 mg to about 3500 mg, about 3500 mg to about 4000 mg, about 4000 mg to about 5000 mg, about 500 mg to about 3000 mg, about 500 to about 1750 mg, about 500 mg to about 1125 mg, about 1125 mg to about 2250 mg, about 1500 mg to about 3000 mg, about 500 mg, about 650 mg, about 750 mg, about 850 mg, about 1000 mg, about 1750 mg, about 2000 mg, or about 3000 mg) on a free base basis of metformin and rapamycin or a pharmaceutically acceptable salt thereof in an amount of from about 0.1 mg to about 25 mg (e.g., about 0.1 mg to about 20 mg, about 0.1 mg to about 18 mg, about 0.1 mg to about 15 mg, about 0.1 mg to about 13 mg, about 0.1 mg to about 10 mg, about 0.1 mg to about 7 mg, about 0.1 mg to about 5 mg, about 0.1 mg to about 3 mg, about 0.1 mg to about 2 mg, about 0.1 mg to about 1 mg, about 0.5 mg to about 20 mg, about 0.5 mg to about 18 mg, about 0.5 mg to about 15 mg, about 0.5 mg to about 13 mg, about 0.5 mg to about 10 mg, about 0.5 mg to about 7 mg, about 0.5 mg to about 5 mg, about 0.5 mg to about 3 mg, about 0.5 mg to about 2 mg, about 0.5 mg to about 1 mg, about 1 mg to about 20 mg, about 1 mg to about 18 mg, about 1 mg to about 15 mg, about 1 mg to about 13 mg, about 1 mg to about 10 mg, about 1 mg to about 7 mg, about 1 mg to about 5 mg, about 1 mg to about 3 mg, about 1 mg to about 2 mg, about 2 mg to about 12 mg, about 4 mg to about 10 mg, about 4 mg to about 8 mg, about 10 mg to about 30 mg, about 13 mg to about 17 mg, about 2 mg to about 4 mg, about 2 mg to about 10 mg, about 1 mg to about 3 mg, about 3 mg to about 7 mg, about 4 mg to about 6 mg, about 15 mg to about 25 mg, about 18 mg to about 22 mg, about 10 mg to about 14 mg, about 0.5 mg, about 1 mg, about 2 mg, about 2.5 mg, about 3 mg, about 4 mg, about 5 mg, about 6 mg, about 7 mg, about 8 mg, about 9 mg, about 10 mg, about 11 mg, about 12 mg, about 13 mg, about 14 mg, or about 15 mg) on a free base basis of rapamycin. The dosage forms can further include a pharmaceutically acceptable carrier and/or an additional therapeutic agent.

In some embodiments, the dosage form includes metformin or a pharmaceutically acceptable salt thereof in an amount of from about 1500 mg to about 3000 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of from about 1500 mg to about 2250 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of from about 2250 mg to about 3000 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of about 1500 mg on a free base basis of metformin and rapamycin or a pharmaceutically acceptable salt thereof in an amount of about 0.5 mg on a free base basis of rapamycin.

In some embodiments, the dosage form includes metformin or a pharmaceutically acceptable salt thereof in an amount of about 3000 mg on a free base basis of metformin and rapamycin or a pharmaceutically acceptable salt thereof in an amount of about 0.5 mg on a free base basis of rapamycin.

In some embodiments, the dosage form includes metformin or a pharmaceutically acceptable salt thereof in an amount of about 1500 mg on a free base basis of metformin and rapamycin or a pharmaceutically acceptable salt thereof in an amount of about 1 mg on a free base basis of rapamycin.

In some embodiments, the dosage form includes metformin or a pharmaceutically acceptable salt thereof in an amount of about 3000 mg on a free base basis of metformin and rapamycin or a pharmaceutically acceptable salt thereof in an amount of about 1 mg on a free base basis of rapamycin.

In some embodiments, the dosage form includes metformin or a pharmaceutically acceptable salt thereof in an amount of from about 500 mg to about 1750 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of from about 500 mg to about 1125 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of from about 1125 mg to about 1750 mg on a free base basis of metformin and rapamycin or a pharmaceutically acceptable salt thereof in an amount of from about 0.5 mg to about 1 mg on a free base basis of rapamycin.

In some embodiments, the dosage form includes metformin or a pharmaceutically acceptable salt thereof in an amount of about 500 mg on a free base basis of metformin and rapamycin or a pharmaceutically acceptable salt thereof in an amount of about 0.5 mg on a free base basis of rapamycin.

In some embodiments, the dosage form includes metformin or a pharmaceutically acceptable salt thereof in an amount of about 1750 mg on a free base basis of metformin and rapamycin or a pharmaceutically acceptable salt thereof in an amount of about 0.5 mg on a free base basis of rapamycin.

In some embodiments, the dosage form includes metformin or a pharmaceutically acceptable salt thereof in an amount of about 500 mg on a free base basis of metformin and rapamycin or a pharmaceutically acceptable salt thereof in an amount of about 1 mg on a free base basis of rapamycin.

In some embodiments, the dosage form includes metformin or a pharmaceutically acceptable salt thereof in an amount of about 1750 mg on a free base basis of metformin and rapamycin or a pharmaceutically acceptable salt thereof in an amount of about 1 mg on a free base basis of rapamycin.

In some embodiments, the weight ratio of metformin to rapamycin is about 10000:1 to about 100:1 (e.g., about 10000:1 to about 300:1, about 5000:1 to about 400:1, about 4000:1 to about 500:1, about 3500:1 to about 750:1, about 3500:1 to about 900:1, about 3500:1 to about 1000:1, about 2500:1 to about 1000:1, about 2500:1 to about 1700:1, about 3000:1 to about 2000:1, about 2000:1 to about 1500:1, about 1300:1 to about 700:1, about 3500:1, about 2500:1, about 1750:1, or about 1000:1). In some embodiments, the weight ratio of metformin to rapamycin is about 3500:1. In some embodiments, the weight ratio of metformin to rapamycin is about 2500:1. In some embodiments, the weight ratio of metformin to rapamycin is about 1000:1. In some embodiments, the weight ratio of metformin to rapamycin is about 1750:1.

Appropriate dosage levels may be determined by any suitable method. Preferably, the active substance is administered at a frequency of 1 to 4 times per day for topical administration, or less often if a drug delivery system is used. Nevertheless, actual dosage levels and time course of administration of the active ingredients in the pharmaceutical compositions of the present disclosure may be varied so as to obtain an amount of the active ingredient which is effective to achieve a desired therapeutic response for a particular patient, composition and mode of administration, without being intolerably toxic to the patient. In certain cases, dosages may deviate from the stated amounts, in particular as a function of age, gender, body weight, diet and general health status of the patient, route of administration, individual response to the active ingredient, nature of the preparation, and time or interval over which administration takes place. Thus, it may be satisfactory in some cases to manage with less than the aforementioned minimum amount, whereas in other cases the stated upper limit may be exceeded. It may in the event of administration of larger amounts be advisable to divide these into multiple individual doses spread over the day.

In some embodiments of the methods disclosed herein, the metformin or a pharmaceutically acceptable salt thereof and the rapamycin or a pharmaceutically acceptable salt thereof is administered orally, parenterally, transdermally, intranasally, sublingually, neuraxially, or ocularly. In some embodiments, the metformin or a pharmaceutically acceptable salt thereof and the rapamycin or a pharmaceutically acceptable salt thereof is administered orally.

The disclosures of all publications cited herein are expressly incorporated herein by reference, each in its entirety, to the same extent as if each were incorporated by reference individually.

### Exemplary Embodiments

Embodiment 1. A method of treating or preventing a neurological, muscular, or proliferative disorder in a subject in need thereof, the method comprising administering a therapeutically effective amount of metformin or a pharmaceutically acceptable salt thereof and rapamycin or a pharmaceutically acceptable salt thereof to the subject; wherein the neurological, muscular, or proliferative disorder is selected from the group consisting of: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), Lafora disease, neuroglioblastoma, and diffuse intrinsic pontine glioma (DIPG).

Embodiment 2. The method of embodiment 1, wherein the neurological, muscular, or proliferative disorder is myotonic dystrophy type 1 (DM1).

Embodiment 3. The method of embodiment 2, wherein after administering the metformin or a pharmaceutically acceptable salt thereof and rapamycin or a pharmaceutically acceptable salt thereof, a reduction in reactive oxygen species (ROS) is measured in the subject.

Embodiment 4. The method of embodiment 1, wherein the neurological, muscular, or proliferative disorder is Duchenne muscular dystrophy (DMD).

Embodiment 5. The method of embodiment 1, wherein the neurological, muscular, or proliferative disorder is spinocerebellar ataxia type 3 (SCA-3).

Embodiment 6. The method of embodiment 1, wherein the neurological, muscular, or proliferative disorder is Alzheimer's disease.

Embodiment 7. The method of embodiment 1, wherein the neurological, muscular, or proliferative disorder is Parkinson's disease.

Embodiment 8. The method of embodiment 1, wherein the neurological, muscular, or proliferative disorder is vascular dementia.

Embodiment 9. The method of embodiment 1, wherein the neurological, muscular, or proliferative disorder is dementia with Lewy bodies (DLB).

Embodiment 10. The method of embodiment 1, wherein the neurological, muscular, or proliferative disorder is Huntington disease (HD).

Embodiment 11. The method of embodiment 1, wherein the neurological, muscular, or proliferative disorder is amyotrophic lateral sclerosis (ALS).

Embodiment 12. The method of embodiment 1, wherein the neurological, muscular, or proliferative disorder is Lafora disease.

Embodiment 13. The method of any one of embodiments 6-12, wherein the method comprises preventing the disorder.

Embodiment 14. The method of embodiment 1, wherein the neurological, muscular, or proliferative disorder is neuroglioblastoma.

Embodiment 15. The method of embodiment 1, wherein the neurological, muscular, or proliferative disorder is diffuse intrinsic pontine glioma (DIPG).

Embodiment 16. The method of any one of embodiments 14-15, wherein after administering the metformin or a pharmaceutically acceptable salt thereof and rapamycin or a pharmaceutically acceptable salt thereof, Akt is activated in the subject.

Embodiment 17. A method of treating or preventing a neurological cancer in a subject in need thereof, the method comprising administering a therapeutically effective amount of metformin or a pharmaceutically acceptable salt thereof and rapamycin or a pharmaceutically acceptable salt thereof to the subject; wherein the cancer is neuroglioblastoma or diffuse intrinsic pontine glioma (DIPG)).

Embodiment 18. The method of embodiment 17, wherein the neurological cancer is neuroglioblastoma.

Embodiment 19. The method of embodiment 17, wherein the neurological cancer is diffuse intrinsic pontine glioma (DIPG).

Embodiment 20. The method of any one of embodiments 17-19, wherein after administering the metformin or a pharmaceutically acceptable salt thereof and rapamycin or a pharmaceutically acceptable salt thereof, Akt is activated in the subject.

Embodiment 21. A method of increasing muscular strength in a subject identified or diagnosed as having muscular weakness, the method comprising administering a therapeutically effective amount of metformin or a pharmaceutically acceptable salt thereof and rapamycin or a pharmaceutically acceptable salt thereof to the subject.

Embodiment 22. A method of preventing or reversing muscle weakness in a subject in need thereof, the method comprising administering a therapeutically effective amount of metformin or a pharmaceutically acceptable salt thereof and rapamycin or a pharmaceutically acceptable salt thereof to the subject.

Embodiment 23. The method of any one of embodiments 2-5 and 21-22, wherein after administering the therapeutically effective amount of metformin or a pharmaceutically acceptable salt thereof and rapamycin or a pharmaceutically acceptable salt thereof to the subject, an increase muscle mass using magnetic resonance imaging (MRI) assessment is measured in the subject.

Embodiment 24. The method of any one of embodiments 2-5 and 21-23, comprising determining an increase in hand grip strength as measured by a dynamometer.

Embodiment 25. The method of any one of embodiments 2-5 and 21-23, comprising determining an at least 10% increase in hand grip strength as measured by a dynamometer.

Embodiment 26. A method of reducing or preventing cellular senescence in a subject, comprising administering a therapeutically effective amount of metformin or a pharmaceutically acceptable salt thereof and rapamycin or a pharmaceutically acceptable salt thereof to the subject.

Embodiment 27. The method of embodiment 26, wherein the method comprises preventing cellular senescence in the subject.

Embodiment 28. A method of increasing motility in a subject, comprising administering a therapeutically effective amount of metformin or a pharmaceutically acceptable salt thereof and rapamycin or a pharmaceutically acceptable salt thereof to the subject.

Embodiment 29. The method of embodiment 28, wherein increasing motility in the subject comprises increasing the number of voluntary muscle contractions in the subject per minute relative to before the therapeutically effective amount of metformin or a pharmaceutically acceptable salt thereof and rapamycin or a pharmaceutically acceptable salt thereof was administered to the subject.

Embodiment 30. The method of any one of embodiments 28-29, wherein increasing motility in the subject comprises increasing the duration of a voluntary muscle contraction in the subject relative to before the therapeutically effective amount of metformin or a pharmaceutically acceptable salt thereof and rapamycin or a pharmaceutically acceptable salt thereof was administered to the subject.

Embodiment 31. The method of any one of embodiments 28-30, comprising determining an increase in the distance walked by the subject during a 10 meter walk test after administering the metformin or a pharmaceutically acceptable salt thereof and rapamycin or a pharmaceutically acceptable salt thereof.

Embodiment 32. The method of any one of embodiments 38-31, comprising determining a reduction in the time taken by the subject to complete a 100 meter walk test after administering the metformin or a pharmaceutically acceptable salt thereof and rapamycin or a pharmaceutically acceptable salt thereof.

Embodiment 33. The method of any one of embodiments 1-32, wherein after administering the metformin or a pharmaceutically acceptable salt thereof and rapamycin or a pharmaceutically acceptable salt thereof, an increase in AMPK activation, inhibition of mTORC1, inhibition of S6 kinase, or any combination thereof are determined in the subject.

Embodiment 34. The method of any one of embodiments 1-33, wherein the metformin or a pharmaceutically acceptable salt thereof and rapamycin or a pharmaceutically acceptable salt thereof are administered orally.

Embodiment 35. The method of any one of embodiments 1-34, wherein the metformin or a pharmaceutically acceptable salt thereof is administered daily.

Embodiment 36. The method of embodiment 35, wherein the dose of metformin or a pharmaceutically acceptable salt thereof is from about 500 mg to about 3000 mg on a free base basis of metformin.

Embodiment 37. The method of any one of embodiments 35-36, wherein the dose of metformin or a pharmaceutically acceptable salt thereof is from about 500 to about 1750 mg on a free base basis of metformin.

Embodiment 38. The method of any one of embodiments 1-37, wherein the rapamycin or a pharmaceutically acceptable salt thereof is administered daily.

Embodiment 39. The method of embodiment 38, wherein the dose of rapamycin or a pharmaceutically acceptable salt thereof is from about 0.1 mg to about 2 mg on a free base basis of rapamycin.

Embodiment 40. The method of any one of embodiments 38-39, wherein the dose of rapamycin or a pharmaceutically acceptable salt thereof is from about 0.5 mg to about 1 mg on a free base basis of rapamycin.

Embodiment 41. The method of any one of embodiments 38-40, wherein the dose of rapamycin or a pharmaceutically acceptable salt thereof is about 0.7 mg on a free base basis of rapamycin.

Embodiment 42. The method of any one of embodiments 1-41, wherein the metformin or a pharmaceutically acceptable salt thereof and rapamycin or a pharmaceutically acceptable salt thereof are administered simultaneously.

Embodiment 43. The method of embodiment 42, wherein the metformin or a pharmaceutically acceptable salt thereof and rapamycin or a pharmaceutically acceptable salt thereof are administered simultaneously as a fixed dosage form.

Embodiment 44. The method of any one of embodiments 1-37, wherein the rapamycin or a pharmaceutically acceptable salt thereof is administered weekly.

Embodiment 45. The method of embodiment 44, wherein the dose of rapamycin or a pharmaceutically acceptable salt thereof is from about 1 mg to about 10 mg on a free base basis of rapamycin.

Embodiment 46. The method of any one of embodiments 44-45, wherein the dose of rapamycin or a pharmaceutically acceptable salt thereof is from about 2 mg to about 10 mg on a free base basis of rapamycin.

Embodiment 47. The method of any one of embodiments 44-46, wherein the dose of rapamycin or a pharmaceutically acceptable salt thereof is about 5 mg on a free base basis of rapamycin.

Embodiment 48. The method of any one of embodiments 1-47, wherein a cyclosporine, tacrolimus, and mycophenolate mofetil were not previously administered to the subject within 1 month of administering the metformin or a pharmaceutically acceptable salt thereof and rapamycin or a pharmaceutically acceptable salt thereof.

Embodiment 49. The method of any one of embodiments 1-48, wherein the subject is not identified or diagnosed as having a disease associated with a kidney.

Embodiment 50. The method of any one of embodiments 1-49, wherein the subject is not identified or diagnosed as having a disease associated with the liver.

Embodiment 51. The method of any one of embodiments 1-50, wherein the subject is not identified or diagnosed as having a disease associated with the heart.

Embodiment 52. The method of any one of embodiments 1-51, wherein the subject is not identified or diagnosed as having diabetes.

Embodiment 53. The method of any one of embodiments 1-52, wherein the subject is not identified or diagnosed as having abnormal endocrine function.

Embodiment 54. The method of any one of embodiments 1-53, wherein the subject was not previously administered a therapeutic agent that modulates the insulin transduction pathway within 1 year of administering the metformin or a pharmaceutically acceptable salt thereof and rapamycin or a pharmaceutically acceptable salt thereof.

### EXAMPLES

### Example 1: Mouse Model Evaluation of Combination Therapy of Metformin and Rapamycin on Neurological, Muscular, and Proliferative Disorders Such as Myotonic Dystrophy Type 1 (DM1).

### Material and Methods:

A DM1 mouse model can be utilized as described in Brockhoff et al., J Clin Invest, 2017, 127 (2): 549-563, which is incorporated by reference in its entirety. The DM1 mouse model that can be used is a transgenic mouse model that overexpresses a mutant human DMPK gene containing a CTG repeat expansion, which is characteristic of DM1. These mice exhibit skeletal muscle weakness and histological hallmarks of DM1, such as muscle fiber atrophy, centralized nuclei, and increased fibrosis. The mice can be bred to obtain DM1 mice that are either wild-type for AMPKα2 (AMPKα2WT) or have a skeletal muscle-specific deletion of AMPKα2 (AMPKα2MKO). The mice can be genotyped by PCR using tail DNA. Insulin resistance, hyperglycemia, glucose uptake and HbAIc can be measured.

In vitro studies can be performed on myoblasts derived from the quadriceps of control and DM1 mice. The myoblasts can be cultured in growth medium, differentiated in differentiation medium, and treated with AICAR or rapamycin. Western blot analysis can be performed to assess the levels of protein expression and phosphorylation of various markers of the AMPK and mTORC1 pathways.

The dosing of the combination therapy can include treating the DM1 mice with the combination therapy metformin and rapamycin to target the deregulated AMPK/mTORC1 pathways in muscle cells. Metformin can be used to activate AMPK, while rapamycin can be used to inhibit mTORC1. The effects of this combination therapy can be assessed by measuring muscle function and gene expression in the treated mice. Doses can be determined based on previous studies, pharmacokinetic data, and toxicity profiles, and can be optimized using various techniques in the art, such as with a dose escalation study. As a non-limiting example, metformin can, for example, be administered at a dose of 300 mg/kg/day in drinking water for 2 weeks, and rapamycin, for example, can be given at 3 mg/kg/day via intraperitoneal injection for 2 weeks.

### Data Analysis:

The efficacy of the drug combination metformin and rapamycin on relevant disease parameters such as blood glucose levels, body weight, insulin sensitivity, or other relevant biomarkers in the DM1 model can be measured by comparing the effect of the drug combination to the effect of each drug alone.

Blood glucose levels: Blood glucose levels can be analyzed by collecting blood samples from the mice at regular intervals and measuring the glucose concentration using a glucose meter or by performing an enzyme-linked immunosorbent assay (ELISA) on the collected serum samples.

Body weight: Body weight can be measured using a digital scale at regular intervals throughout the study. The data can then be analyzed using statistical methods such as ANOVA or linear regression to determine any significant differences between the treatment groups.

Insulin sensitivity: Insulin sensitivity can be assessed using an insulin tolerance test (ITT) or glucose tolerance test (GTT). These tests involve administering insulin or glucose to the mice and measuring the resulting changes in blood glucose levels. The data can then be analyzed using statistical methods such as ANOVA or linear regression to determine any significant differences between the treatment groups.

Other relevant biomarkers: Other relevant biomarkers can be analyzed by collecting blood samples or tissue samples from the mice at the end of the study and performing various assays such as ELISA, Western blotting, or PCR. The data can then be analyzed using statistical methods such as ANOVA or linear regression to determine any significant differences between the treatment groups.

The degree of drug synergy can be assessed using quantitative methods such as the combination index (CI) or the Bliss independence model. These models allow for an accurate determination of the extent to which the drug combination metformin and rapamycin synergizes in the treatment of myotonic dystrophy type I. To validate the results of the in vivo experiment, additional experiments such as dose-response curves, time course studies, or genetic manipulations can be performed.

Example 2: Microfluidic-Based Assay for Automated In Vivo Lifespan and Healthspan Assessment of Rapamycin and Metformin and their Combinations Using the Nematode Caenorhabditis elegans

### SUMMARY

### Objectives

The goal of this study was to investigate the role of rapamycin (referred to herein as “Compound 1” or “Cpd 1”), metformin (referred to herein as “Compound 2” or “Cpd 2”), and their combination on lifespan and healthspan on C. Elegans. For this purpose, a microfluidic-based technology was used.

### Overview of the Method

For the purpose of this study, the two compounds and four combinations of the two compounds were tested on a microfluidic platform. Worms were injected at the L4 larval stage, confined in dedicated microfluidic chambers, and continuously subjected to the presence of the two compounds, alone or in combination, from the L4 larval stage to the end of the experiment (corresponding to the day when the worm is dead). Acquisition of short sequences of videos for each microfluidic chamber containing worms were executed automatically every six (6) hours during the duration of the assay. The measurement of the motility, as well as the worm size to address compound toxicity (1) and the reproduction assessing longevity (2), were performed on the acquired sequences of videos by using an algorithm (3,4). Dead/live worms were identified automatically, based on the motility parameters. Each condition was tested in four technical replicates on different microfluidic cartridges. The experiment was performed once.

### Study Design and Methods

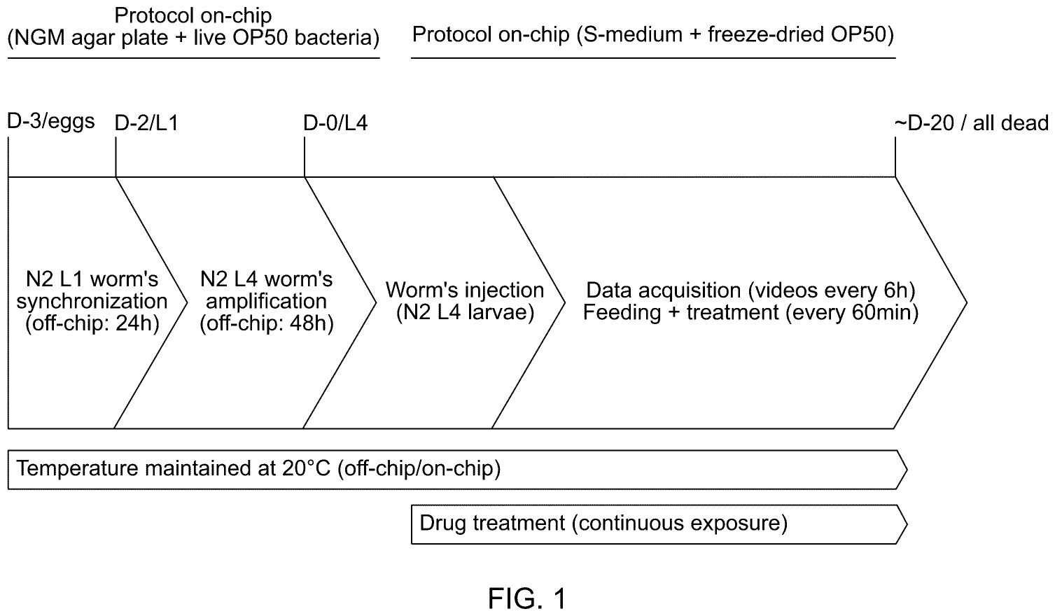

The proposed study was conducted according to the design and timeline depicted in FIG. 1. N2 wild-type C. elegans were grown on solid nematode growth medium (NGM) agar plates until the adult stage and harvested in complete S-Medium. Eggs from the adult population were hatched overnight in S-Medium and the resulting L1 progeny was recovered with a dedicated filter. L1 larvae were then collected through filtering in S-medium, seeded on NGM agar plates and grown for 48 hours (h) at 20° C. After 48 h, the worm population had reached the L4 larval stage. L4 larvae were then collected through filtering in 5 mL S-medium. 20 to 30 L4 were injected in the microfluidic chips for each condition. Worms were then continuously fed on-chip via bacterial medium, for the duration of the entire experiment. Videos of each micro-chamber were acquired after the feeding during the whole experiment, every 6 hours.

### Preparation of the Tested Substances

The two compounds, Compound 1 and Compound 2, were suspended in DMSO at the stock concentration of 100 mM and 2.5 M, respectively (Table 1). Compounds were then mixed with the bacterial medium at the targeted concentrations, alone or in combination (Table 2). For all tested conditions, the final vehicle (DMSO) concentration was 1%. For the negative control condition, nematodes were fed with a bacterial medium containing only the vehicle solution (DMSO 1%). All stock solutions were stored at −20° C. and dilutions were made freshly on the day of starting the on-chip experiment and then every 72 h to replace the compound/bacterial medium solutions.

TABLE 1
List of the chemicals used in the present study.

| Chemicals | Provider | Solvent | Concentration stock |
|---|---|---|---|
| Compound 1 | ElixiRA AG | DMSO | 100 mM |
| Compound 2 | ElixiRA AG | DMSO | 2.5M |

TABLE 2
Conditions tested in the present study.

| Compound 1 | Compound 2 | ID condition |
|---|---|---|
| 0 | 0 | DMSO 1% |

| | | | |
|---|---|---|---|
| 100 | μM | 0 | Cpd 1 |

| | | | |
|---|---|---|---|
| 0 | 25 | mM | Cpd 2 |

| | | | | |
|---|---|---|---|---|
| 100 | μM | 25 | mM | Combo A |
| 33 | μM | 25 | mM | Combo B |
| 100 | μM | 8.5 | mM | Combo C |
| 50 | μM | 12.5 | mM | Combo D |

### Bacteria Preparation

Worms were continuously fed on-chip via bacterial medium (freeze-dried E. coli OP50 in complete S-medium) at the concentration of 8.05E+9 cells per milliliter. Briefly, 500 mg of freeze-dried OP50 were dissolved in 5 mL of complete S-medium and carefully homogenized using vortexing. The bacterial concentration was monitored using a spectrophotometer and adjusted to 8.05E+9 if needed (5).

### Worm Preparation

Eggs of C. elegans (N2 wild-type strain, provided by the Caenorhabditis Genetics Center) were hatched on solid nematode growth medium (NGM) agar plates that were seeded with E. coli OP50 and grown in this condition for 4 days at 20° C. After 4 days, a mix-stage of N2 was present on the NGM agar plates, including mainly adult worms and eggs. The worms' population was harvested and filtered in complete S-medium to separate adult worms from the other mix-stage populations. The isolated adult worms were incubated overnight at room temperature. L1 larvae, which had hatched from the eggs laid by the adult worms, were then collected through filtering in S-medium, seeded on NGM agar plates and grown for 48 h at 20° C. After 48 h, the worm's population had reached the L4 larval stage. L4 larvae were then collected through filtering in 5 mL h, the initial solutions of bacteria and compounds were replaced by new solutions containing bacteria. 4 μL of fresh food/compounds solution were injected into the microfluidic chip per each tested condition every 60 min. Videos of each micro-chamber were acquired after the feeding.

### Phenotypic Analysis

Five time-resolved phenotypic readouts were extracted during the assays, including:
Worm size (area): this parameter was measured to evaluate the potential effects of the tested compound on growth retardation or larval arrest phenotype (7).
Sexual maturity: this parameter was measured to evaluate the potential effects of the tested compound on the timing required by a worm to reach the sexual maturity, determined by the observation of the first embryo (i.e., egg) produced in each microfluidic chamber. This phenotype was also indicative for a general sterility phenotype (i.e., no egg produced) (7).
Worm fertility: this parameter was measured to evaluate the potential effects of the tested compound on the average number of eggs produced by each worm during the reproductive span, calculated via the images acquired during the assay (7).
Reproductive span: this parameter was measured to evaluate the potential effects of the tested compound on the duration in which the worms have reproductive ability, determined by the observation of the last embryo produced in each microfluidic chamber.
Worm longevity: this parameter was measured to evaluate the potential effects of the tested compound on the worm's survival.

Worm motility is an additional phenotypic readout, where five parameters of the motility were assessed every 6 hours from acquired videos (2 seconds long, 10 frames per second for each video). At each time point, the values recorded for dead worms were excluded. The five motility parameters monitored included (3):
the amplitude of the movement at the head
the amplitude of the movement at the middle of the worm body
the amplitude of tail movement
the worm bending frequency
the worm velocity

These motility parameters were analyzed by phases (Area Under the Curve calculation following by an 2-way ANOVA tests), as defined below:
Phase 1: from day 1 to day 5
Phase 2: from day 6 to day 10
Phase 3: from day 11 to day 15
Phase 4: from day 16 to day 20

### Statistics

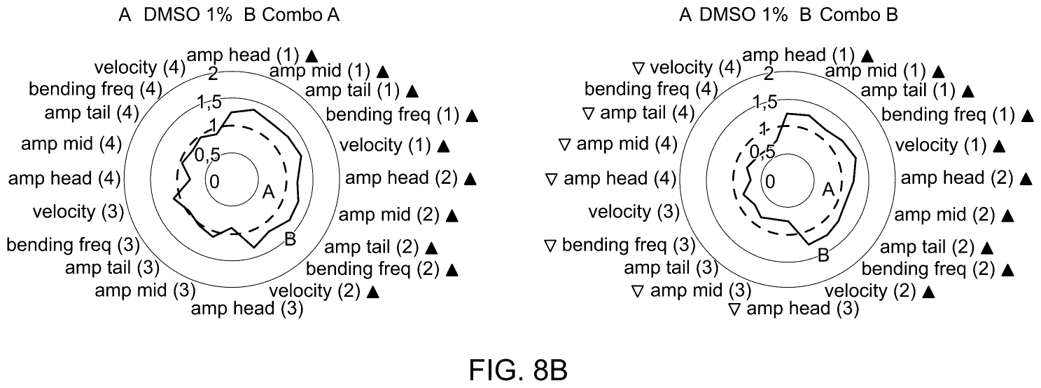

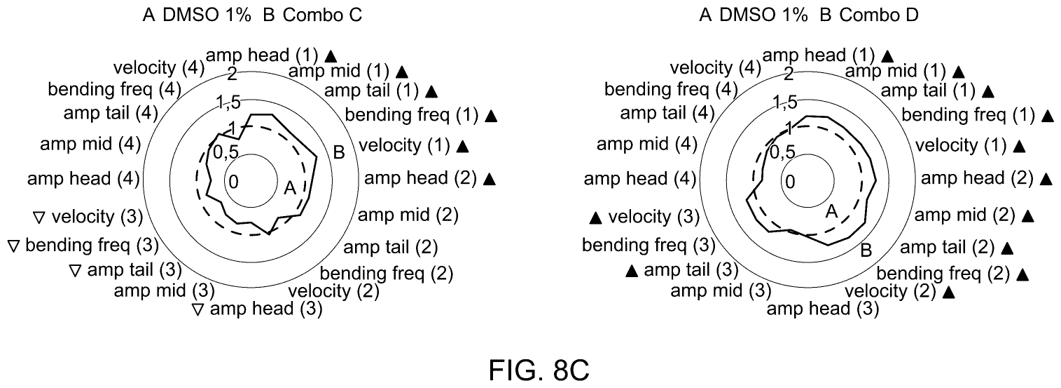

Raw values were normalized and compared to their respective negative control (when indicated). Survival analyses were performed using the Kaplan-Meier method and the significance of differences between survival curves calculated using the log rank test. To compare the interaction between groups, two-way ANOVA tests were performed. Analysis of variance, assessed by Bonferroni multiple comparison test, was used when comparing more than two groups. GraphPad Prism 5 (GraphPad Software) was used for all other statistical analyses including calculation of sd, standard error of mean (sem) and area under the curve (AUC). All p values <0.05 were considered significant. The radar charts shown in FIGS. 8A-8C were: ² statistics versus the negative control (DMSO 1%).

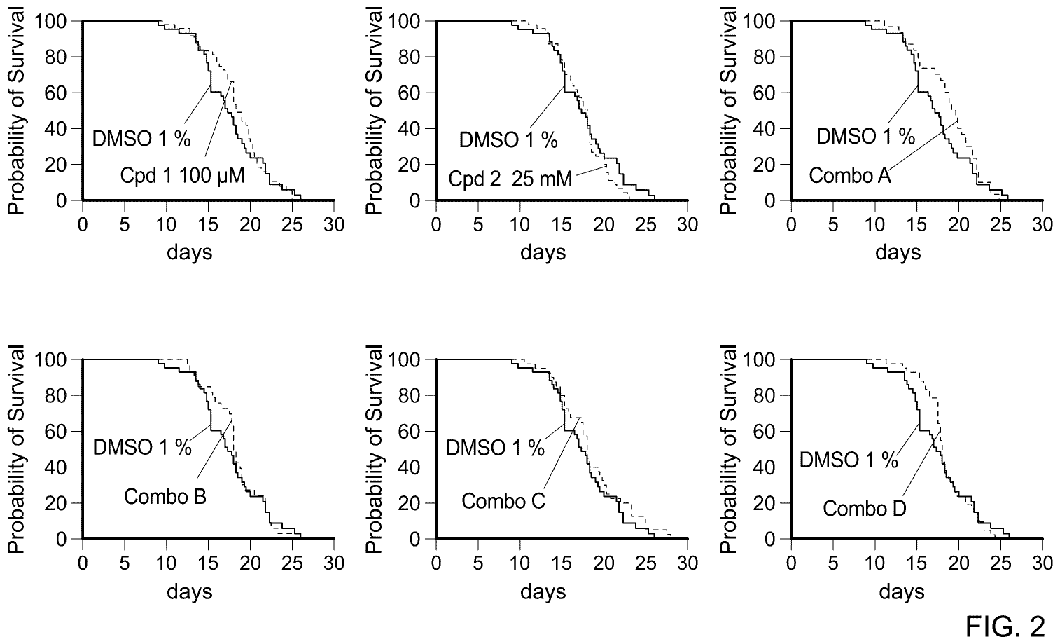

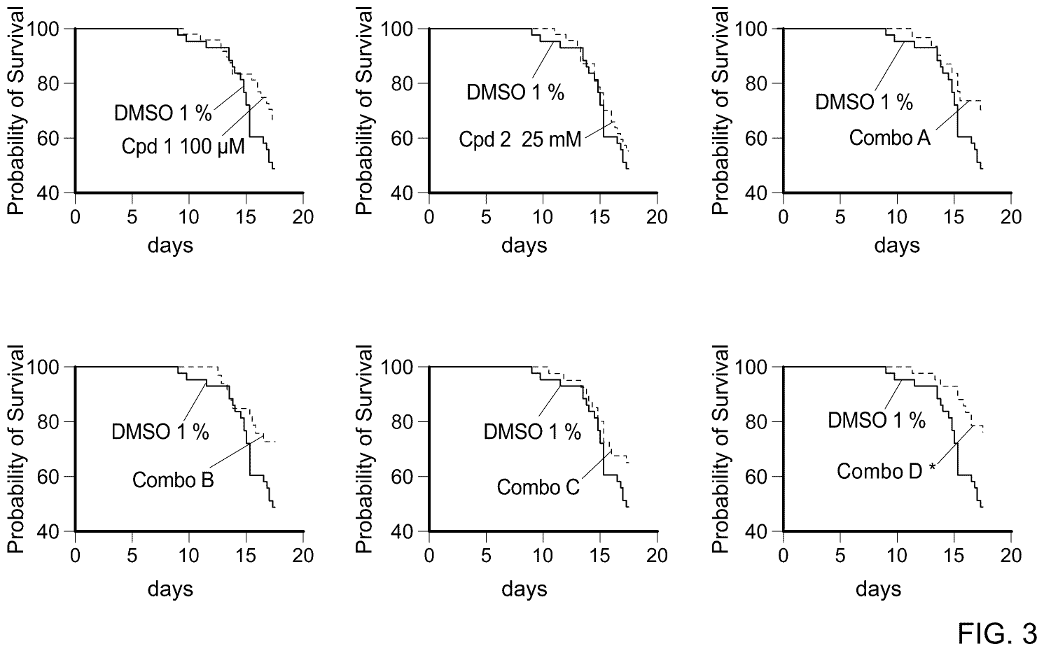

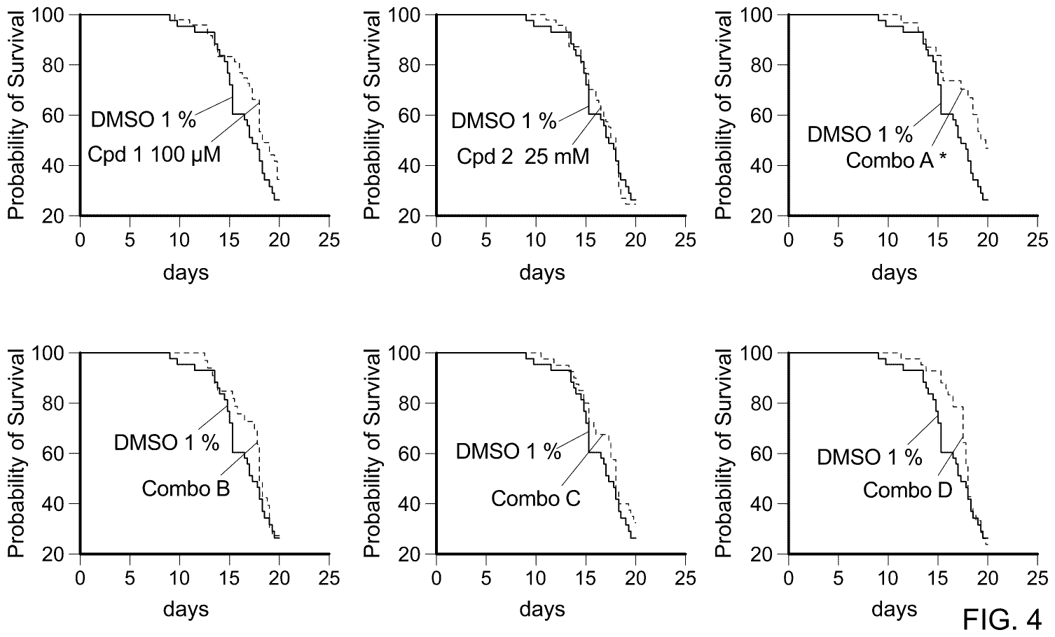

Summary and interpretation of results from life span monitoring:
Although all compounds either tested alone or in combination had a prolonging effect on survival with e.g., Combo D+14% when compared to negative control, none of the tested conditions reached significance under current conditions on overall survival of the N2 worms. (FIG. 2 and Table 3).
Combo D treatment showed a significant effect (+56.0%; p<0.05) on the 50% worm's survival when compared to the negative control (FIG. 3 and Table 4). Cpd 1, Combo A and Combo B treatments displayed a moderate improvement of the 50% worm's survival, but not significantly (0.05<p<0.1)
Combo A treatment showed a significant effect (+77.8%; p<0.05) on the 25% worm's survival when compared to the negative control (FIG. 4 and Table 5).

### Reproduction and Growth Monitoring

### Observations:

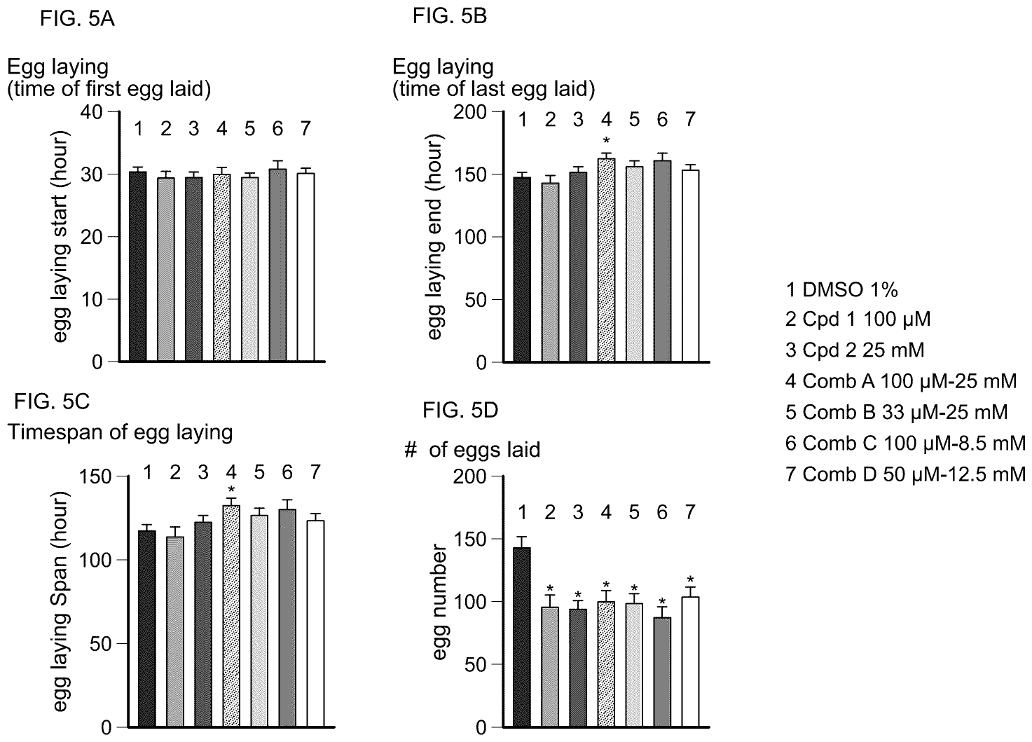

FIGS. 5A-5D are bar graphs of the time (in hours) from injection of the L4 larvae until production of the first (FIG. 5A) and last (FIG. 5B) egg, as well as the timespan of egg laying (FIG. 5C) and the average number of eggs laid per worm within the timespan of egg laying (FIG. 5D), observed in N2 wild type worms treated with with the conditions summarized in Table 2. Bar graphs represent mean+/−sem. p values were obtained by comparing the negative control (blue bars) versus the treatment via One-way ANOVA followed by Bonferroni's multiple comparisons test (p<0.05). * statistics versus the negative control (DMSO 1%). Tables 6-9 summarize the data from FIGS. 5A-5D.

TABLE 6
Summary of the results from FIG. 5A.

| | Egg laying | | | Variation | p value |
|---|---|---|---|---|---|
| Conditions | start (hours) | sd | n^{c} | (%) | (vs neg.) |
| DMSO 1% | 30.57 | 4.27 | 54 | | |
| Cpd 1 | 29.59 | 4.93 | 32 | −3.2 | >0.9999 |
| Cpd 2 | 29.63 | 4.08 | 33 | −3.1 | >0.9999 |
| Combo A | 30.16 | 5.06 | 31 | −1.3 | >0.9999 |
| Combo B | 29.65 | 3.35 | 35 | −3.0 | >0.9999 |
| Combo C | 31.00 | 5.29 | 21 | +1.4 | >0.9999 |
| Combo D | 30.30 | 4.43 | 46 | −0.9 | >0.9999 |

n^{c} corresponds to the number of chambers in which egg laying start was detected. Within the chamber egg-laying start was determined at the population level and not at the individual worm level.

TABLE 7
Summary of the results from FIG. 5B.

| | Egg laying | | | Variation | p value |
|---|---|---|---|---|---|
| Conditions | end (hours) | sd | n^{c} | (%) | (vs neg.) |
| DMSO 1% | 148.6 | 22.7 | 54 | | |
| Cpd 1 | 144.0 | 28.6 | 32 | −3.1 | >0.9999 |
| Cpd 2 | 152.7 | 19.1 | 33 | +2.8 | >0.9999 |
| Combo A | 163.3 | 20.4 | 31 | +9.9 | 0.0273* |
| Combo B | 157.0 | 22.5 | 35 | +5.7 | 0.5513 |
| Combo C | 161.8 | 22.9 | 21 | +8.9 | 0.1503 |
| Combo D | 154.4 | 22.4 | 46 | +3.9 | >0.9999 |

n^{c} corresponds to the number of chambers in which egg laying start was detected. Within the chamber egg-laying start was determined at the population level and not at the individual worm level.

TABLE 8
Summary of the results from FIG. 5C.

| | Reproductive | | | | |
|---|---|---|---|---|---|
| | span | | | Variation | p value |
| Conditions | (hours) | sd | n^{c} | (%) | (vs neg.) |
| DMSO 1% | 118.0 | 22.6 | 54 | | |
| Cpd 1 | 114.4 | 29.9 | 32 | −3.1 | >0.9999 |
| Cpd 2 | 123.1 | 19.3 | 33 | +4.3 | >0.9999 |
| Combo A | 133.1 | 20.5 | 31 | +12.8 | 0.0241* |
| Combo B | 127.3 | 21.4 | 35 | +7.9 | 0.3915 |
| Combo C | 130.8 | 23.2 | 21 | +10.8 | 0.1939 |
| Combo D | 124.1 | 23.7 | 46 | +5.2 | >0.9999 |

n^{c} corresponds to the number of chambers in which egg laying start was detected. Within the chamber egg-laying start was determined at the population level and not at the individual worm level.

TABLE 9
Summary of the results from FIG. 5D.

| | | | | Variation | p value |
|---|---|---|---|---|---|
| Conditions | Eggs laid (#) | sd | n^{c} | (%) | (vs neg.) |
| DMSO 1% | 143.9 | 57.2 | 54 | | |
| Cpd 1 | 96.6 | 50.1 | 32 | −32.9 | <0.0001 |
| Cpd 2 | 94.9 | 34.4 | 33 | −34.1 | <0.0001 |
| Combo A | 100.8 | 44.3 | 31 | −30.0 | 0.0003 |
| Combo B | 99.5 | 40.1 | 35 | −30.9 | 0.0001 |
| Combo C | 88.1 | 35.7 | 21 | −38.8 | <0.0001 |
| Combo D | 104.7 | 47.8 | 46 | −27.2 | 0.0002 |

n^{c} corresponds to the number of chambers in which egg laying start was detected. Within the chamber egg-laying start was determined at the population level and not at the individual worm level.

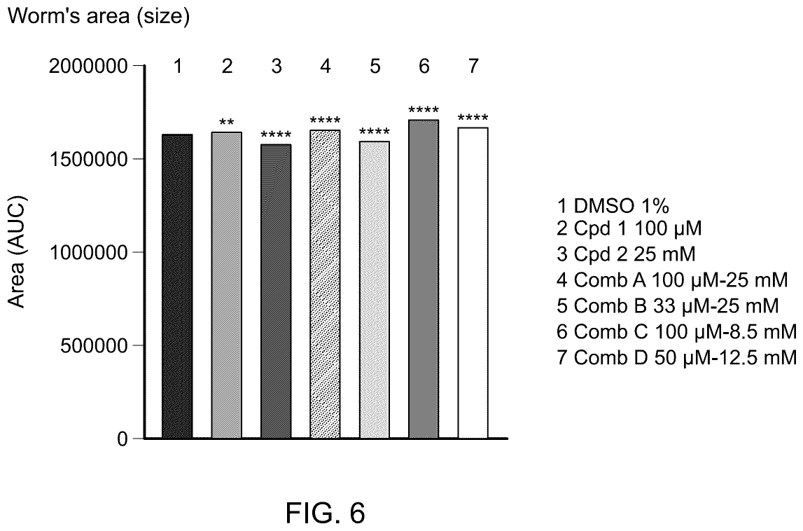

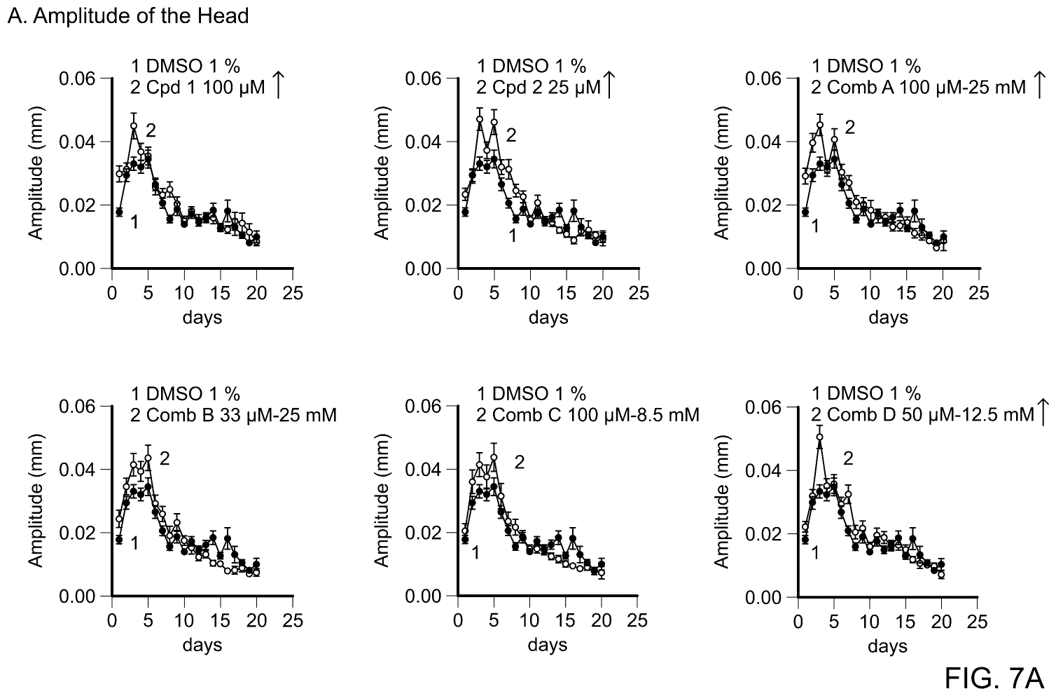

FIG. 6 is a bar graph of the worm's growth (area under the curves) of N2 wild type worms treated with with the conditions summarized in Table 2. p values were obtained by comparing the negative control (blue line) versus the treatment via 2-way ANOVA to assess overall curve differences followed by Bonferroni's multiple comparisons test. **p<0.01; ***p<0.001; ****p<0.0001. Table 10 summarizes the data from FIG. 6.

TABLE 10
Summary of the results related to worm area shown in FIG. 6.

| | | Timespan | | p value |
|---|---|---|---|---|
| Conditions | AUC | analyzed (days) | Variation (%) | (vs neg.) |
| DMSO 1% | 1641164 | 20 | | |
| Cpd 1 | 1654770 | 20 | +0.8 | 0.0065 |
| Cpd 2 | 1587229 | 20 | −3.3 | <0.0001 |
| Combo A | 1664410 | 20 | +1.4 | <0.0001 |
| Combo B | 1603469 | 20 | −2.3 | <0.0001 |
| Combo C | 1718405 | 20 | +4.7 | <0.0001 |
| Combo D | 1677401 | 50 | +2.2 | <0.0001 |

Summary and interpretation of the results from reproduction and growth monitoring:
All the tested conditions (compounds tested alone or in combination) significantly reduced the number of eggs laid per worms when compared to the negative control (FIG. 5A and Table 9).
Combo A treatment slightly impacted (+9.9%, p<0.05) the time of the last egg laid and as a result extended the overall timespan of egg laying by 12.8% (p<0.05) when compared to the negative control (FIGS. 5B-5C and Table 8).
All the tested conditions (compounds tested alone or in combination) significantly impacted the worm's size when compared to the negative control (FIG. 6 and Table 10): Cpd 1, Combo A, Combo C and Combo D treatments showed a significant improvement on the worm's size, while Cpd 2 and Combo B displayed a significant decreased on the worm's size when compared to the negative control (FIG. 6 and Table 10).

### Motility Monitoring

### Observations:

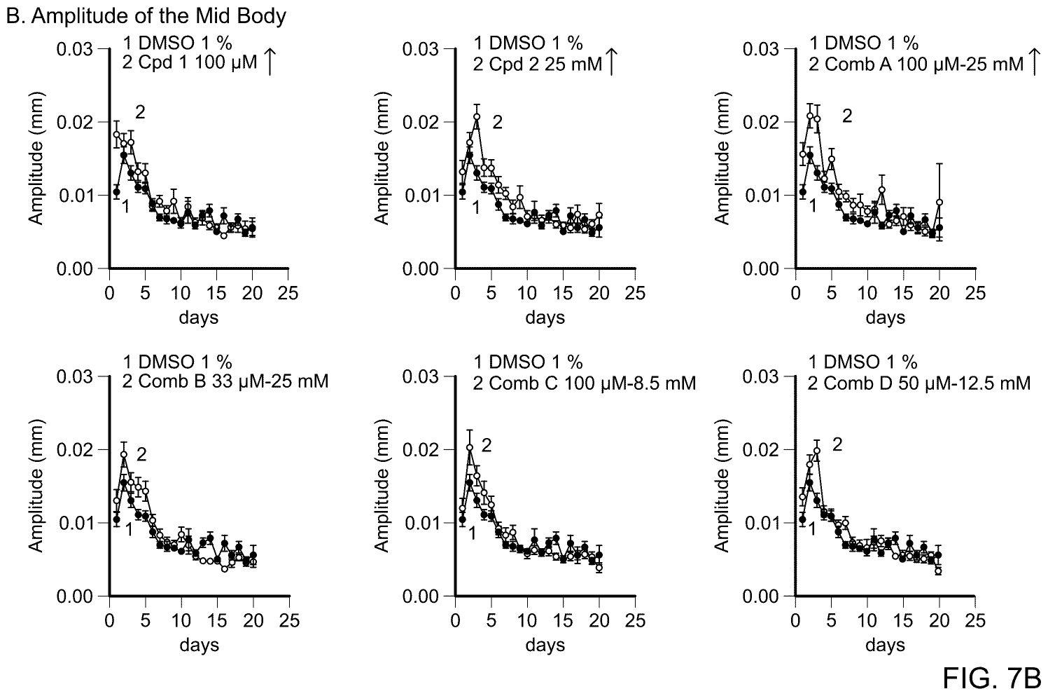

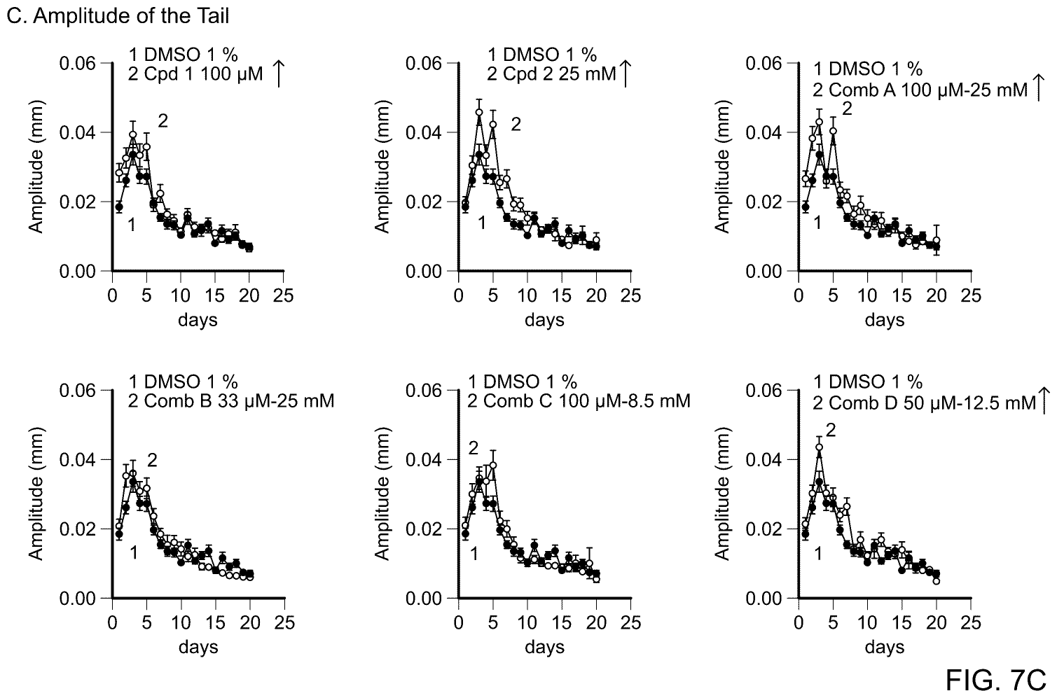

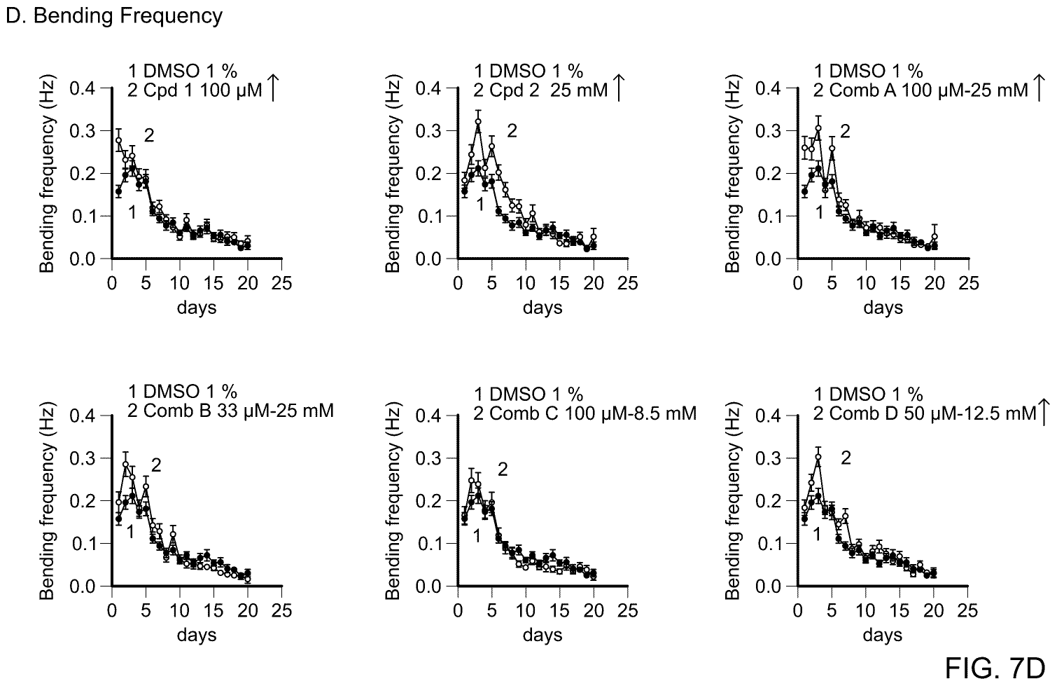

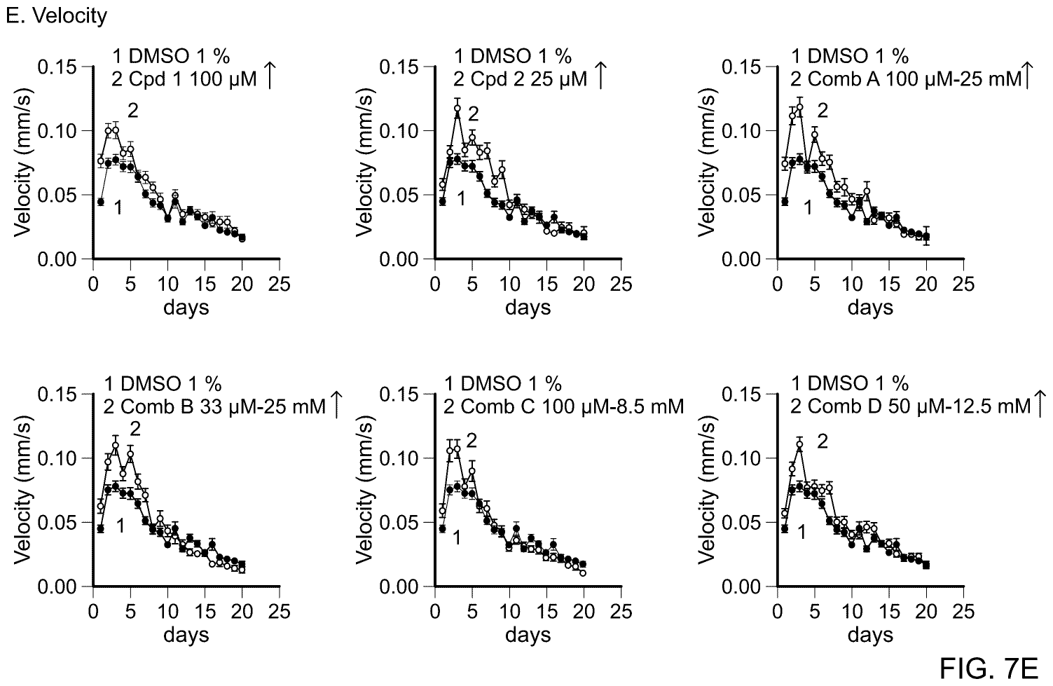

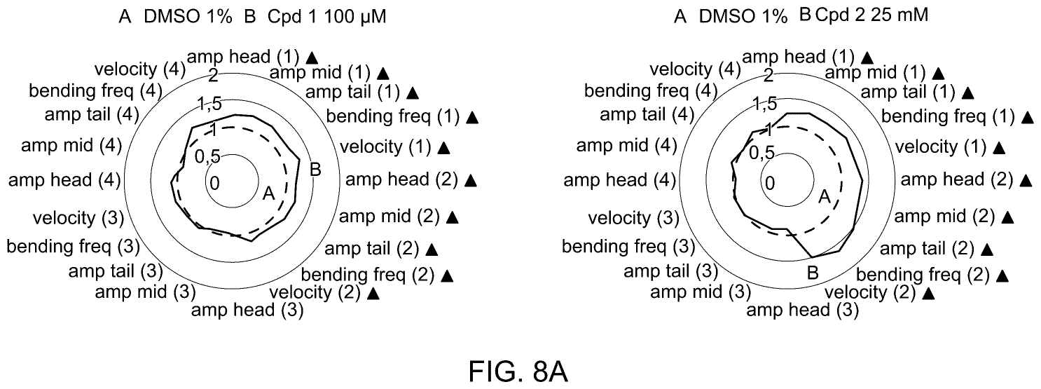

FIGS. 7A-7D are plots of the worm's motility (FIG. 7A, amplitude of the head; FIG. 7B, amplitude of the midbody; FIG. 7C, amplitude of the tail; FIG. 7D, bending frequency; FIG. 7E, velocity) of N2 wild type worms treated with with the conditions summarized in Table 2. p values were obtained by comparing the negative control (blue line) versus the treatment via 2-way ANOVA to assess overall curve differences followed by Bonferroni's multiple comparisons (p<0.05). Error bars indicate sem. ↑: significant increase vs the negative control (curve shown in blue) for the phenotype analyzed (2-way ANOVA curve comparison (p<0.05)) ↓: significant decrease vs the negative control (curve shown in blue) for the phenotype analyzed (2-way ANOVA curve comparison (p<0.05)).

FIGS. 8A-8C are a series of radar charts of the worm's motility (amplitude of the head, amplitude of the midbody, amplitude of the tail, velocity and bending frequency) at different phases (as indicated clockwise around each radar chart phases denoted by the number “1” correspond to phases DO to D5; phases denoted by the number “2” correspond to phases D6 to D10; phases denoted by the number “3” correspond to phases D11 to D15; phases denoted by the number “4” correspond to phases D16-D20), of N2 wild type worms treated with Cpd 1 (final concentration of 100 μM) and Cpd 2 (final concentration of 25 mM) (FIG. 8A); Combo A (Cpd 1 100 μM+Cpd 2 25 mM) and Combo B (Cpd 1 33 μM+Cpd 2 25 mM) (FIG. 8B); Combo C (Cpd 1 100 μM+Cpd 2 8.5 mM) and Combo D (Cpd 1 50 μM+Cpd 2 12.5 mM) (FIG. 8C). Values represented on the radar charts correspond to the ratio over the negative control (DMSO 1%) of the AUC computed from the curves shown in the FIG. 7. ↓: significant decrease vs the negative control (DMSO 1%, blue line) for the phenotype analyzed (2-way ANOVA curve comparison (p<0.05)); ↑: significant increase vs the negative control (DMSO 1%, blue line) for the phenotype analyzed (2-way ANOVA curve comparison (p<0.05)).

Summary and Interpretation of the Results from Monitoring Motility and Behavior During Basal Conditions:
The overall motility of the worms treated with Cpd 1 (all motility parameters improved by 14% in average), Cpd 2 (all motility parameters improved by 22% in average), Combo A (all motility parameters improved by 19% in average), Combo B (1 of the 5 motility parameters improved by 14%) and Combo D (4 of the 5 motility parameters improved by 16% in average) was significantly increased when compared to the negative control (FIGS. 7A-7E). No effect on the motility was observed when the worms were treated with Combo C (FIGS. 7A-7E).
The overall improvement of the worm's motility in the population treated with Cpd 1, Cpd 2 and Combo A (FIGS. 7A-7E) was explained by a significant increase in almost all the motility parameters during Phase 1 (D0 to D5) and Phase 2 (D6 to D10) only (FIGS. 8A-8C). No effect on the motility was observed in Phase 3 (D11 to D15) and Phase 4 (D16 to D20).
The overall improvement of the worm's motility in the population treated with Combo D (FIGS. 7A-7E) by 16% in average was explained by a significant increase in most of the motility parameters during Phase 1 (D0 to D5), Phase 2 (D6 to D10) and Phase 3 (D11 to D15) (FIGS. 8A-8C). No effect on the motility was observed in Phase 4 (D16 to D20).
Combo B treatment showed a significant improvement of all the motility parameters in Phase 1 (D0 to D5) and Phase 2 (D6 to D10) (FIGS. 8A-8C). However, motility was significantly impaired in later phases (Phase 3 and Phase 4), explaining the weak overall effect on the motility for this specific condition (FIGS. 7A-7E).
Combo C treatment showed a significant improvement of all the motility parameters in Phase 1 (D0 to D5) only (FIGS. 8A-8C). The motility was significantly impaired in Phase 3 (D11 to D15), explaining the overall absence of effect on the motility for this specific condition (FIGS. 7A-7E).

### CONCLUSION

### Conclusion of the Study

The biological effects of Cpd 1 and Cpd 2 and their combinations on aging and related age-associated phenotypes were analyzed and profiled. Overall, this study suggests that the combination of Cpd 1 and Cpd 2 at high doses (100 μM and 25 mM respectively, corresponding to Combo A) and half of the maximum doses (50 μM and 12.5 mM respectively, corresponding to Combo D) have the abilities to provide significant synergistic effects on lifespan and motility in the nematode C. elegans. Further conclusions are summarized below:
Combo D (Cpd 1 50 μM+Cpd 2 12.5 mM) treatment showed a significant effect (+56.0%; p<0.05) on the 50% worm's survival when compared to the negative control, while no impact was observed later in life (25% worm's survival). Furthermore, Combo A (Cpd 1 100 μM+Cpd 2 25 mM) treatment showed a significant effect (+77.8%; p<0.05) on the 25% worm's survival when compared to the negative control, while a moderate improvement (p<0.1) of the 50% worm's survival was observed. In conclusion, a synergistic effect was observed for Combo A and Combo D on the survival, as no significant effect was monitored with the 2 compounds tested alone. These results show that a combination of rapamycin and metformin synergistically elongate the lifespan of C. elegans.
In general, while Combo A displayed a tendency to improve the overall worm's lifespan (+14.4%; p=0.2), none of the tested conditions had a significant effect on the worm's whole survival when compared to the negative control.
All the tested conditions significantly reduced the number of eggs laid per worm when compared to the negative control. No synergistic effect was observed when the two compounds were tested in combination (i.e., the combinations did not lead to a reduction in the number of eggs laid per worm). Without wishing to be bound by theory, it is believed that some interventions, especially caloric restriction, promote beneficial effects on longevity while reducing the ability to reproduce across species (2). Some compounds, known to mimic caloric restriction effect, also triggered such an effect on the reproduction.
However, Combo A treatment slightly impacted (+9.9%, p<0.05) the time of the last egg laid and extended the overall timespan of egg laying by 12.8% (p<0.05) when compared to the negative control, which can be interpreted as having a beneficial effect on the reproductive system of C. elegans.
Regarding the worm's size, Cpd 1, Combo A, Combo C, and Combo D treatments showed a significant improvement on the worm's size, while Cpd 2 and Combo B displayed a significant decrease on the worm's size when compared to the negative control. Based on these observations, it appeared that Cpd 2 at high doses negatively impacted the worm's size (alone or in combination as observed in Combo B): Cpd 1 at the high dose (100 μM) had the ability to revert this deleterious effect on size, as observed in the Combo A condition (+4.9% increase versus Cpd 2 alone at 25 mM).
The overall motility of the worms treated with Cpd 1, Cpd 2, Combo A, Combo B, and Combo D was significantly increased when compared to the negative control. Interestingly, the overall improvement of the worm's motility in the population treated with Cpd 1, Cpd 2, and Combo A was explained by a significant increase in almost all the motility parameters during Phase 1 (D0 to D5) and Phase 2 (D6 to D10) only. No effect on the motility was observed in Phase 3 (D11 to D15) and Phase 4 (D16 to D20) for these specific conditions. Furthermore, the overall improvement of the worm's motility in the population treated with Combo D was explained by a significant increase in most of the motility parameters during Phase 1 (D0 to D5), Phase 2 (D6 to D10) and Phase 3 (D11 to D15). This observation indicated that Combo D treatment has the ability to maintain and improve the fitness and motility of the worms later in life.
Furthermore, while some deleterious effects on worm's motility were observed in Combo B during late phases of life (Phase 3 and 4), these negative effects were reverted in Combo A and in Combo D in which Cpd 2 concentration was decreased (Combo D) or Cpd 1 increased (Combo A).

Overall, this study suggests that the combination of Cpd 1 and Cpd 2 at high doses (100 μM and 25 mM, respectively) and half of the maximum doses (50 μM and 12.5 mM, respectively) have the abilities to provide significant synergistic effects on both increasing lifespan and improving motility in the nematode C. elegans.

### REFERENCES

1) Hunt P R. The C. elegans model in toxicity testing. J Appl Toxicol. 2017 January; 37 (1)
2) Moatt J P et al. The effect of dietary restriction on reproduction: a meta-analytic perspective. BMC Evol Biol. 2016 Oct. 7; 16 (1): 199
3) Atakan H B et al. Automated Platform for Long-Term Culture and High-Content Phenotyping of Single C. elegans Worms. Sci Rep. 2019 Oct. 4; 9 (1): 14340
4) Atakan H B et al. Automated high-content phenotyping from the first larval stage till the onset of adulthood of the nematode Caenorhabditis elegans. Lab Chip. 2018 Dec. 18; 19 (1): 120-135
5) Gao A W et al. High-content phenotypic analysis of a C. elegans recombinant inbred population identifies genetic and molecular regulators of lifespan. bioRxiv [Preprint]. 2024 Jan. 16:2024.01.15.575638
6) Cornaglia et al. Automated longitudinal monitoring of in vivo protein aggregation in neurodegenerative disease C. elegans models. Mol Neurodegener. 2016 Feb. 9; 11:17.
7) Mouchiroud L et al. Worm-on-Chip technology as a new alternative for early toxicity studies. Toxicology Letters. 2021 350, S118-S119.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating or preventing a neurological or muscular disorder in a subject in need thereof, the method comprising administering a therapeutically effective amount of metformin or a pharmaceutically acceptable salt thereof and rapamycin or a pharmaceutically acceptable salt thereof to the subject; wherein the neurological or muscular is selected from the group consisting of: myotonic dystrophy type 1 (DM1), Duchenne muscular dystrophy (DMD), spinocerebellar ataxia type 3 (SCA-3), Alzheimer's disease, Parkinson's disease, vascular dementia, dementia with Lewy bodies (DLB), Huntington disease (HD), amyotrophic lateral sclerosis (ALS), and Lafora disease.

2. The method of claim 1, wherein the neurological or muscular disorder is myotonic dystrophy type 1 (DM1).

3. (canceled)

4. The method of claim 1, wherein the neurological or muscular disorder is Duchenne muscular dystrophy (DMD).

5. (canceled)

6. The method of claim 1, wherein the neurological or muscular disorder is Alzheimer's disease.

7. The method of claim 1, wherein the neurological or muscular disorder is Parkinson's disease.

8.-10. (canceled)

11. The method of claim 1, wherein the neurological or muscular disorder is amyotrophic lateral sclerosis (ALS).

12. (canceled)

13. The method of claim 1, wherein the method comprises preventing the disorder.

14.-25. (canceled)

26. A method of reducing or preventing cellular senescence in a subject, comprising administering a therapeutically effective amount of metformin or a pharmaceutically acceptable salt thereof and rapamycin or a pharmaceutically acceptable salt thereof to the subject.

27. The method of claim 26, wherein the method comprises preventing cellular senescence in the subject.

28. A method of increasing motility in a subject, comprising administering a therapeutically effective amount of metformin or a pharmaceutically acceptable salt thereof and rapamycin or a pharmaceutically acceptable salt thereof to the subject.

29.-30. (canceled)

31. The method of claim 28, comprising determining an increase in the distance walked by the subject during a 6 minute walk test (6MWT) after administering the metformin or a pharmaceutically acceptable salt thereof and rapamycin or a pharmaceutically acceptable salt thereof.

32.-33. (canceled)

34. The method of claim 1, wherein the metformin or a pharmaceutically acceptable salt thereof and rapamycin or a pharmaceutically acceptable salt thereof are administered orally.

35. The method of claim 1, wherein the metformin or a pharmaceutically acceptable salt thereof is administered daily.

36. The method of claim 35, wherein the dose of metformin or a pharmaceutically acceptable salt thereof is from about 500 mg to about 3000 mg on a free base basis of metformin.

37. (canceled)

38. The method of claim 1, wherein the rapamycin or a pharmaceutically acceptable salt thereof is administered daily.

39. The method of claim 38, wherein the dose of rapamycin or a pharmaceutically acceptable salt thereof is from about 0.1 mg to about 10 mg on a free base basis of rapamycin.

40.-41. (canceled)

42. The method of claim 1, wherein the metformin or a pharmaceutically acceptable salt thereof and rapamycin or a pharmaceutically acceptable salt thereof are administered simultaneously.

43. The method of claim 42, wherein the metformin or a pharmaceutically acceptable salt thereof and rapamycin or a pharmaceutically acceptable salt thereof are administered simultaneously as a fixed dosage form.

44. The method of claim 1, wherein the rapamycin or a pharmaceutically acceptable salt thereof is administered weekly.

45. The method of claim 44, wherein the dose of rapamycin or a pharmaceutically acceptable salt thereof is from about 1 mg to about 10 mg on a free base basis of rapamycin.

46.-54. (canceled)

55. The method of claim 1, wherein:
the metformin or a pharmaceutically acceptable salt thereof is administered daily at a dose of about 500 mg to about 3000 mg on a free base basis of metformin; and
the rapamycin or a pharmaceutically acceptable salt thereof is administered daily at a dose of about 0.1 mg to about 10 mg on a free base basis of rapamycin.

56. The method of claim 1, wherein:
the metformin or a pharmaceutically acceptable salt thereof is administered weekly at a dose of about 500 mg to about 3000 mg on a free base basis of metformin; and
the rapamycin or a pharmaceutically acceptable salt thereof is administered weekly at a dose of about 1 mg to about 10 mg on a free base basis of rapamycin.
